(19) United States
(12) Patent Application Publication
(10) Pub. No.: US 2016/0024575 A1
(43) Pub. Date: Jan. 28, 2016

(54) CIRCULATING SMALL NONCODING RNA MARKERS

(71) Applicant: The Regents of the University of California, Oakland, CA (US)

(72) Inventors: Stephen Spindler, Riverside, CA (US); Joseph M. Dhahbi, Riverside, CA (US)

(73) Assignee: The Regents of the University of California, Oakland, CA (US)

(21) Appl. No.: 14/268,848

(22) Filed: May 2, 2014

Related U.S. Application Data
(60) Provisional application No. 61/818,869, filed on May 2, 2013.

(51) Int. Cl. C12Q 1/68

(52) U.S. Cl. CPC C12Q 1/6876; C12Q 1/6886; C12Q 2600/178; C12Q 2600/166

(57) ABSTRACT

This application describes small noncoding RNA markers that can be found in a biological sample taken from an individual. The level of such markers are useful for determining the individual's health status, especially in comparison with others. Methods and kits for the use of these markers are provided as well.

### CROSS-REFERENCES TO RELATED APPLICATIONS

This application claims the benefit of U.S. Provisional Application No. 61/818,869, filed May 2, 2013, the contents of which are hereby incorporated by reference in their entirety for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file “Sequence Listing for 81906-907996 (217410US).txt”, created on Dec. 22, 2014 and containing 3,336 bytes, machine format IBM-PC, MS-Windows operating system is hereby incorporated by reference in its entirety for all purposes.

### BACKGROUND OF THE INVENTION

Small noncoding RNAs (sncRNAs) mediate a variety of cellular functions in animals and plants. It has been discovered using deep sequencing that sncRNAs circulate in the blood of humans and other mammals. The most abundant types of circulating sncRNAs are microRNAs (miRNAs), 5′ transfer RNA (tRNA) halves, and YRNA fragments, with minute amounts of other types. It has been suggested that some sncRNAs are specifically processed and secreted as macromolecular complexes to protect the non-coding RNAs from degradation.

Properties of circulating sncRNAs are consistent with a possible role as signaling molecules. For instance, it has been shown that circulating miRNAs can enter cells and regulate cellular functions.

5′ tRNA halves are derived from a small subset of tRNAs, implying that they are produced by tRNA type-specific biogenesis and/or release. The 5′ tRNA halves are not in exosomes or microvesicles, but circulate as particles of 100-300 kDa. The size of these particles suggest that the 5′ tRNA halves are a component of a macromolecular complex; this is supported by the loss of 5′ tRNA halves from serum or plasma treated with EDTA, a chelating agent, but their retention in plasma anticoagulated with heparin or citrate. A survey of somatic tissues reveals that 5′ tRNA halves are concentrated within blood cells and hematopoietic tissues, but scant in other tissues, suggesting that they may be produced by blood cells.

Full-length YRNAs are small (84-112 nt) RNAs with poorly characterized functions, best known because they make up part of the Ro ribonucleoprotein autoantigens in connective tissue diseases. The present inventors have discovered YRNA fragments of lengths 27 nt and 30-33 nt, derived from the 5′ ends of specific YRNAs, and generated by cleavage within a predicted internal loop. These 5′ YRNA fragments make up a large proportion of all small RNAs (including miRNAs) present in human serum. They are also present in plasma, are not present in exosomes or microvesicles, and circulate as part of a complex with a mass between 100 and 300 kDa.

Studies have also shown that sncRNAs may server as markers of health and disease states. For example, serum levels of specific sncRNAs such as 5′ tRNA halves change markedly with age. Additionally, caloric restriction can mitigate these age-related changes, thereby indicating that sncRNA levels are under physiologic control. The inventors have discovered that levels of circulating tRNA-derived and YRNA-derived fragments correlate to the presence of breast cancer.

There is a need in the pertinent field for non-invasive methods for detection of healthy and disease states, including various types of cancer, such as breast cancer. There is also a need for measuring circulating small noncoding RNAs. The present invention satisfies these needs and provides related advantages as well.

### BRIEF SUMMARY OF THE INVENTION

The present invention is based, in part, on the discovery of two types of small noncoding RNA molecules (5′ tRNA halves and YRNA fragments) found in the circulating blood (e.g., serum or plasma) of a mammal (e.g., human). The 5′ tRNA halves are derived from the 5′ end of a subset of tRNAs and correspond to the first 27, 28, 29, 30, 31, 32, 33, 34, or even 35 nucleotides of a tRNA gene sequence (e.g., any one of those named in Table 3 or Table 4). They are found in serum as particles of about 100-300 kDa, being a part of a macromolecular structure (e.g., in complex with one or more proteins) but not in exosomes or microvesicles. These 5′ tRNA halves are also found within blood cells and hematopoietic tissues, indicating their origin as being produced by blood cells. The inventors observed that the serum levels of these small RNAs change markedly, either increase or decrease, with age (see, e.g., Table 3 or Table 4), and that such change can be mitigated by calorie restrictions.

The second type of small noncoding RNA molecules identified by the inventors are YRNAs. They are small (84-112 nt) RNAs that correspond to the first 27 or 30-33 nucleotides of a YRNA gene sequence (e.g., any one of those named in Table 5 or provided herein). They make up part of the Ro ribonucleoprotein autoantigens in connective tissue diseases. In surveying small RNAs present in the serum of healthy adult humans, the inventors have discovered YRNA fragments that are derived from the 5′ ends of specific YRNAs which were previously either annotated as pseudogenes or predicted informatically. There fragments are generated by cleavage within a predicted internal loop. The 5′ YRNA fragments provided herein make up a large proportion of all small RNAs (including miRNAs) present in human serum. They are also present in plasma, but are not in exosomes or microvesicles. Like, 5′ tRNA halves, YRNA fragments circulate as part of a complex with a mass between 100 and 300 kDa.

The inventors have observed that the serum levels of these small RNAs can increase or decrease with the presence of cancer such as breast cancer, aging and caloric restriction. As such, the present invention provides novel markers and non-invasive means for monitoring an individual's health status such as aging, potential longevity, and presence/risk of disease such as cancer, infectious diseases, cardiovascular diseases, neurodegenerative disorders including Alzheimer's disease, Huntington's disease, etc., especially in comparison with one or more other individuals with known health/aging/caloric intake status.

In the first aspect, the present invention provides novel polynucleotides (e.g., small RNA molecules) that each corresponds to a section of a tRNA having the polynucleotide sequence of the first 27, 28, 29, 30, 31, 32, 33, 34, or 35 nucleotides starting from the 5′ end of the tRNA sequence, or a complement thereof. Table 3 and 4 provide a list of these tRNAs. The invention also provides polynucleotide sequences that are complementary to the small RNA sequences, as such complementary sequences can be useful for detecting these small RNA molecules.

In the second aspect, the present invention provides novel polynucleotides (e.g., small RNA molecules) that each corresponds to a section of a YRNA having the polynucleotide sequence of the first 27, 28, 29, 30, 31, 32, 33, 34, or 35 nucleotides starting from the 5′ end of the YRNA sequence, or a complement thereof. Table 5 provides a list of these YRNAs. The invention also provides polynucleotide sequences that are complementary to the small RNA sequences, as such complementary sequences can be useful for detecting these small RNA molecules.

In the third aspect, the present invention provides a polynucleotide probe including a tRNA half or YRNA fragment described herein and a detectable moiety. The snRNA can be conjugated (e.g., linked) to the detectable label.

In the fourth aspect, the present invention provides a kit for detecting a polynucleotide having the nucleotide sequence corresponding to the first 27, 28, 29, 30, 31, 32, 33, 34, or 35 nucleotides starting from the 5′ end of the a tRNA provided in Table 3 or Table 4, or a YRNA provided in Table 5, or the complement thereof. The kit in some cases includes appropriate primters for amplifying a tRNA half or YRNA fragment as described herein. The kit may also contain a control that provides a sample of the polynucleotide or a complement thereof and the polynucleotide probe described above. As the kit may beused for diagnositic purposes as described herein, in some embodiments, the kit may further include a standard control in which the target tRNa half or YRNA fragment of the kit is at a concentration of a known state of health/age/caloric intake.

In another aspect, the present invention provides an expression cassette (e.g., expression vector) that includes a promoter, e.g., a heterologous promoter, that is operably linked to the polynucleotide described herein. The expression cassette can be introduced (e.g., transformed or transfected) into a host cells such as a eukaryotic cell or a prokaryotic cell. Alternatively, the expression cassette can be introduced into a stable cell line. In some embodiments, the expression cassette is introduced into a human cell.

In yet another aspect, the present invention provides a method for quantitating a polynucleotide having a nucleotide sequence corresponding to the first 27, 28, 29, 30, 31, 32, 33, 34, or 35 nucleotides of a tRNA provided in Tables 3 or 4, or a YRNA provided in Table 5; or a complement thereof. The method includes extracting nucleic acids (e.g., RNA) from a biological sample and measuring the level of the polynucleotide in the extract. In some cases, the step of measuring comprises an amplification reaction. In other cases, the step of measuring comprises sequencing. The biological sample can be whole blood, serum, plasma, saliva, mucus, urine, cerebrospinal fluid, nipple fluid, or another bodily fluid. Optionally, the biological sample can be a tissue sample such as breast tissue, hematopoietic tissue and lymphoid tissue, from, e.g., a biopsy.

In another aspect, the present invention provides a method for determining or monitoring the health status of a mammal based on the level of at least one polynucleotide or complement thereof in a biological sample taken from the mammal (e.g., a human patient). The method includes quantitating at least one polynucleotide has a nucleotide sequence corresponding to the first 27, 28, 29, 30, 31, 32, 33, 34, or 35 nucleotides of a tRNA provided in Table 3 or Table 4 or a YRNA gene provided in Table 5; or a complement thereof in the sample. The method also includes comparing the level to that of a control sample and concluding that the health status of the mammal is better or worse than the control if the level of the polynucleotide(s) is greater than that of the control sample. In some cases, the mammal is a human being. In some cases, the health status is aging status and/or predicted longevity. In some cases, the health status is caloric intake, especially in relation with caloric consumption by the mammal (e.g., after subtraction of the number of calories consumed due to physical/physiological activity during the same time period). In other instances, the health status is the risk or presence of breast cancer. In some cases, the health status is the presence or risk of certain diseases, for example, various types of cancer, infectious diseases, cardiovascular diseases, neurodegenerative disorders including but not limited to Alzheimer's disease, Huntington's disease, etc. In some cases, the biological sample is blood, serum, or plasma. In other cases, the biological sample is blood cells and hematopoietic tissues (e.g., leukocytes). Depending on the specific small RNA marker, as shown in Table 3, an increase or decrease can indicate a relatively better/improved health status or more restricted calorie intake. An increase or a decrease in the level of the specific small RNA marker, as shown in Tables 4 and 5, can indicate the presence of breast cancer. Once a diagnosis is made that a subject being tested has or is at risk of later developing a disorder among those named above, the subject should be given treatment for the disorder or regularly monitored for the onset of the disorder such that preventive and/or therapeutic measures can be taken as appropriate.

Typically, the determining and monitoring is based on comparing the level of one or more small RNA molecules found in a biological sample taken from a mammal (e.g., a human) with the level of the same small RNA marker(s) found in the same type of tissue or cell sample taken from another mammal (i.e., a control subject of the same species, often the same gender, with known age and health status, such as predicted longevity, presence/absence/risk of certain diseases, and caloric intake over consumption) to establish a comparison in terms of an increased or decreased level, which in turn provides indication of more or less advanced aging process, better or worse disease state/risk, in relation to the control subject. In some cases, the monitoring is achieved by comparing the levels of one or more small RNA marker(s) in the same individual's samples taken at two or more different times to establish a comparison, and the detected increase/decrease (or lack thereof) will indicate the changes (or lack thereof) in the individual's health status during the period marked by the times when the samples were taken. Once a conclusion is reached regarding the individual's health status, either comparing with a control subject or comparing with the individual him/herself at an earlier time, additional steps in terms of therapeutic and preventive measures may be taken to remedy any undesirable effects, such as by changing caloric intake/consumption, changing life style to prevent/minimize risk of certain diseases, staring treatment for conditions such as cancer or neurodegenerative diseases, or maintaining a routine of regular medical examination for early detection and intervention of any relevant medical conditions.

In yet another aspect, the present invention provides a kit for determining or monitoring the health status of a mammal (e.g., a human). The kit contains agents for detecting one or more small RNA markers (e.g., those having a nucleotide sequence corresponding to the first 30-35 nucleotides of the tRNA listed in Tables 3 and 4, or those having a nucleotide sequence corresponding to the first 27-35 nucleotides of the YRNA listed in Table 5.), such as by performing an amplification reaction (e.g., polymerase chain reaction or PCR and reverse transcription polymerase chain reaction or RT-PCR) to identify the RNA marker. In some cases, the agent for detection may include the polynucleotide probe described above. The kit may also contain a standard control sample, which provides the standard value(s) of the marker(s) from a particular tissue/cell sample from a subject of known health status such as aging, disease presence/risk, and caloric intake (in relation to caloric consumption). Optionally, an instruction manual is also provided in the kit.

### BRIEF DESCRIPTION OF THE DRAWINGS

FIG. 1A-FIG. 1C show the length distribution of reads obtained by deep sequencing of small RNAs extracted from mouse serum. Shown here are only those reads that map to the mm10 (GRCm38) mouse genome. FIG. 1A shows length distribution is displayed by abundance of sequencing reads combined from 9 serum samples. Reads were mapped to the mouse genome with bowtie according to Maq's default policy, either allowing (blue bars) or disallowing (red bars) multiple reportable alignments for each read. FIG. 1B shows combined reads were mapped to the mouse genome with bowtie according to the end-to-end k-difference policy, either allowing (blue bars) or disallowing (red bars) multiple reportable alignments for each read. FIG. 1C shows length distribution of separate sequencing reads obtained from 9 individual serum small RNA samples. Length distribution is displayed by abundance of sequencing reads that were mapped to the mouse genome with bowtie according to Maq's default policy, and allowing multiple reportable alignments for each read. Bars with different colors denote the source of the sequenced serum small RNA from the 9 different samples.

FIG. 2A-FIG. 2C show and annotation of combined sequencing reads of small RNAs extracted from 9 mouse serum samples. FIG. 2A shows length distribution by abundance of the total reads mapped to the mouse genome (black bars), with reads annotated as mapping to either miRNAs (red bars), tRNAs (blue bars), rRNAs (green bars), or other small RNAs (yellow bars). Other small RNAs include scRNA, snRNA, and srpRNA. Note that the reads in the 20-24 nt and the 30-33 nt peaks are almost exclusively annotated as miRNAs and tRNAs, respectively. FIG. 2B shows a pie chart showing the percentage of reads mapping to the specific types of small RNAs. FIG. 2C shows frequencies of 5′ tRNA half types represented in the aligned reads.

FIG. 3A-FIG. 3B show UCSC genome browser screenshots illustrating alignment of reads to two tRNA genes. Shown are the Illumina sequencing reads (red), and the tRNA genes (blue) as annotated in the tRNA genes track “Transfer RNA genes identified with tRNAscan-SE”. FIG. 3A shows the alignment (number of reads, y-axis) shows that all the sequencing reads align to the 5′ end of chr1.tRNA704-GlyGCC gene. FIG. 3B shows the majority of the sequencing reads align to the 5′ end of chr11.tRNA945-ArgCCG gene, whereas only a very small number of sequencing reads aligns to the 3′ end.

FIG. 4 shows cleavage sites of tRNAs. The cloverleaf structure of chr1.tRNA704-GlyGCC gene (SEQ ID NO:1) showing cleavage sites at the anticodon loop, with the percentage of the reads that map to the 5′ end of the tRNA (arrowheads). The cleavage sites are upstream of the GCC anticodon located at nucleotides 33 to 35. Numbers inside the anticodon loop indicate anticodon nucleotides positions

FIG. 5A-FIG. 5G show detection of 5′ tRNA halves in mouse serum. Northern blot analysis of RNA extracted from U2OS cells cultured in the absence (−) or presence (+) of sodium arsenite (AS), or from 0.4 ml of mouse serum. The blot was hybridized to a ³²P-end-labeled oligonucleotide probe complementary to the 5′ (FIG. 5A) or 3′ end (FIG. 5B) of tRNA-Gly-GCC. The blot was also hybridized to a ³²P-end-labeled oligonucleotide probe complementary to the 5′ (FIG. 5C) or 3′ end (FIG. 5D) of tRNA-Val-CAC. 5′ tRNA halves were also detected in fractionated mouse serum. (FIGS. 5E-F) Northern blotting analysis was carried out on RNA extracted from either 0.4 ml of mouse whole serum, from the supernatant (Sup) after ultracentrifugation of 0.4 ml of mouse serum at 110,000 g, or the ultracentrifugation pellet. The blot was hybridized to a ³²P-end-labeled oligonucleotide probe complementary to the 5′ (FIG. 5E) or 3′ end (FIG. 5F) of tRNA-Gly-GCC. FIG. 5G shows that ultrafiltration indicates a size for tRNA serum particles between 100 and 300 kDa. Samples of 0.2 ml serum mixed with 1.8 ml PBS were subjected to ultrafiltration through Vivaspin 2 columns with 30, 100, and 300 kDa MW cut-offs. Total RNAs were extracted from filtrate (f) and concentrate (c) fractions. Blot was hybridized to ³²P-end-labeled oligonucleotide probes complementary to the 5′ end of tRNA-Gly-GCC. The positions of full length tRNAs and tRNA halves are indicated on the right. DM: decade markers.

FIG. 6A-FIG. 6B show Northern blotting analysis of tRNA-Val-CAC halves in mouse serum. FIG. 6A shows RNA extracted from 0.4 ml of mouse whole serum, from supernatant or pellet after ultracentrifugation of 0.4 ml of mouse serum at 11x0000 g and analyzed with northern blotting by hybridization to a ³²P 5′-end labeled oligonucleotide probe complementary to the 5′ end of tRNA-Val-CAC. FIG. 6B shows RNA extracted from 0.2 ml serum mixed with 1.8 ml PBS subjected to ultrafiltration with Vivaspin 2 columns with 30, 100, and 300 kDa MW cut-off. Total RNAs were extracted from filtrate (f) and concentrate (c) fractions. Blot was hybridized to a ³²P 5′-end labeled oligonucleotide probe complementary to the 5′ end of tRNA-Val-CAC. The positions of full length tRNAs and tRNA halves are indicated on the right. DM: decade markers.

FIG. 7A-FIG. 7F show tissue distribution of tRNA-Gly-GCC halves. Northern blotting analysis of RNA extracted from the indicated mouse tissues. Blots were hybridized with ³²P-end-labeled oligonucleotide probes complementary to the 5′ (FIGS. 7A, C, and E) or 3′ end (FIGS. 7B, D, and F) of tRNA-Gly-GCC. The positions of full length tRNAs and tRNA halves are indicated on the right. DM: decade markers.

FIG. 8A-FIG. 8E show tissue distribution of tRNA-Val-CAC halves. Northern blotting analysis of RNA extracted from the indicated mouse tissues. Blots were hybridized with ³²P-end-labeled oligonucleotide probes complementary to the 5′ (FIGS. 8A, B, and D) or 3′ end (FIGS. 8C and E) of tRNA-Val-CAC. The positions of full length tRNAs and tRNA halves are indicated on the right. DM: decade markers.

FIG. 9 shows detection of 5′ tRNA halves in mouse plasma and serum. Northern blot of RNAs extracted from 0.4 ml of mouse serum, serum treated with EDTA, and heparin- or EDTA-collected plasma, hybridized to a ³²P 5′-end labeled oligonucleotide probe complementary to the 5′ end of tRNA-Gly-GCC. EDTA sharply lowers the abundance of 5′ tRNA halves in serum; in plasma, 5′ tRNA halves are abundant when heparin is the anticoagulant, but are nearly absent when EDTA is present. 5′ tRNA halves are similarly abundant when calcium citrate is used as the anticoagulant (not shown).

FIG. 10A-FIG. 10C show real-time PCR amplification plots of circulating miRNAs. Shown are the amplification plots for miR-16 (blue), miR-24 (black), and the spiked-in miR-Cel-39 (red) measured in mouse serum (FIG. 10A), mouse serum treated with EDTA (FIG. 10B), and mouse plasma collected on EDTA (FIG. 10C). The y-axis represents the relative fluorescence units (RFU) in a semi-log scale. The x-axis represents the cycle at which fluorescence was detected above an automatically determined threshold for the indicated miRNA. EDTA does not change the concentration of miRNAs in plasma.

FIG. 11 shows 5′ tRNA halves are also present in human serum and leukocytes, but not in EDTA plasma. RNAs were extracted from human leukocytes, 0.4 ml of human serum, or EDTA-collected plasma. The Northern blot was hybridized to a ³²P 5′-end labeled oligonucleotide probe complementary to the 5′ end of tRNA-Gly-GCC. The positions of full length tRNAs and tRNA halves are indicated on the right. DM: decade markers.

FIG. 12 shows length distribution of sequencing reads that mapped to the RepeatMasker classes of DNA, LINE, LTR, Low_complexity, RC, SINE, Satellite, and Simple_repeat. Read length distribution is displayed by abundance of sequencing.

FIG. 13A-FIG. 13B show the scarcity of 5′ tRNA-Asn halves in mouse serum. Northern blot analysis of RNA extracted from U2OS cells cultured in the absence (−) or presence (+) of sodium arsenite (AS), or from 0.4 ml of mouse serum, or from the supernatant (Sup) after ultracentrifugation of 0.4 ml of mouse serum at 110000 g. The blot was hybridized to a ³²P-end-labeled oligonucleotide probe complementary to the 5′ end of tRNA-Gly-GCC (FIG. 13A) or the 5′ end of tRNA-Asn-GTT (FIG. 13B). The blot hybridized to the 5′ end of tRNA-Gly-GCC was exposed to an X-ray film for 25 minutes, while the blot hybridized to the 5′ end of tRNA-Asn was exposed for 5 days. The positions of full length tRNAs and tRNA halves are indicated on the right. DM: decade markers.

FIG. 14A-FIG. 14E show the length distribution and annotation of small RNAs in human serum. FIG. 14A: Length distribution of reads obtained by deep sequencing of small RNAs extracted from human serum. Shown here are only those reads that map to the hg19 (GRCh37) human genome, with length distribution plotted against abundance of sequencing reads. Sequencing reads combined from five different human serum samples were mapped to the human genome with Bowtie according to the end-to-end k-difference policy with two mismatches, either allowing (blue bars) or disallowing (red bars) multiple reportable alignments for each read. FIG. 14B: Sequencing reads from the five individual human serum samples shown as a pool in (FIG. 14A). Bars with different colors denote the source of the sequenced human serum small RNA from the five individual samples. Distributions in the five individual samples are similar. FIG. 14C: Length distribution of annotated reads obtained by deep sequencing of small RNAs extracted from the five human serum samples. Length distribution is plotted against abundance of the reads annotated as miRNAs, YRNAs, tRNAs, rRNAs, or other sRNAs (snRNAs and snoRNAs). FIG. 14D: A pie chart showing the percentage of reads from the five pooled samples mapping to the indicated specific types of small RNAs. FIG. 14E: Frequencies of YRNA types represented in the aligned reads. YRNAs are classified in Ensembl as RNY1, RNY3, RNY4, RNY5, pseudogenes originating from the four human YRNAs (RNY1P, RNY3P, RNY4P, RNY5P), and a group of predicted YRNAs from the Rfam database.

FIG. 15A-FIG. 15E show characterization of circulating 5′ YRNA fragments. FIG. 15A: UCSC genome browser screenshots illustrating alignment of reads to YRNAs from Ensembl GRCh37 release 70. Shown are the Illumina sequencing reads (blue) aligning to the ENST00000516507 transcript encoded by the RNY4 gene (upper panel) and the ENST00000362735 transcript encoded by the RNY4P24 pseudogene (lower panel). Also shown are the Gene Annotations from ENCODE/GENCODE Version 14, and a custom track (YRNAs) depicting the coding strand. The alignment (number of reads, y-axis) shows that the majority of the sequencing reads align to the 5′ end of the YRNA. FIG. 15B: Important features of predicted YRNA secondary structure (SEQ ID NO:2), cleavage sites, and frequencies. The schematic structure of YRNAs was produced by Varna (6) from the RF00019 Y_RNA Family. The ‘conservation’ option was chosen as the coloring scheme for the secondary structure. The 5′ and 3′ ends are indicated. The putative cleavage sites at the predicted internal loop are denoted by arrows, with the percentage of the reads that map to the 5′ ends of YRNAs. FIG. 15C-FIG. 15D: Northern blot analysis of RNA extracted from human serum or plasma, and from U2OS cells. The blot was hybridized to 32P-end-labeled oligonucleotide probes complementary to the 5′ (C) or 3′ (D) ends of RNY4. Lanes 1-3: RNA extracted from 0.2 ml of whole serum, EDTA plasma (Plasma-E) or heparin plasma (Plasma-H). Lanes 4-7: Samples of 0.2 ml serum mixed with 1.8 ml PBS were subjected to ultrafiltration through Vivaspin 2 columns with 100 and 300 kDa MW cut-offs. Total RNAs were extracted from filtrate (f) and concentrate (c) fractions after the ultrafiltration step. Lanes 8-9: RNAs extracted from U2OS cells (CON), and U2OS cells treated with UV (UV). FIG. 15E: Northern blotting analysis of RNA extracted from either 0.2 ml of whole serum (Whole), from the supernatant (Sup) after ultracentrifugation of 0.2 ml of serum at 110,000 g, or the ultracentrifugation pellet (Pellet). The blot was hybridized to a 32P-end-labeled oligonucleotide probe complementary to the 5′ end of RNY4. The positions of full length YRNAs and YRNA fragments are indicated on the right. M: decade markers.

FIG. 16A-FIG. 16E show comparison of read length and annotation of sequencing reads from human serum and EDTA plasma. Serum: red; Plasma: blue. FIG. 16A: Length distribution of all reads mapping to the hg19 (GRCh37) human genome is displayed by abundance of sequencing reads from serum and EDTA plasma prepared from blood drawn from the same individual. Reads were mapped to the human genome with Bowtie according to the end-to-end k-difference policy with zero mismatches and allowing multiple reportable alignments for each read. FIG. 16B: miRNAs map to reads in the 20-24 nt peak in both serum and plasma. The x-axis represents the read length in nucleotides. The y-axis represents the reads of the indicated length as the percentage of the total reads sequenced from the human sample (serum or plasma). FIG. 16C: YRNAs reads make up the 27 nt peak in both serum and plasma. FIG. 16D: YRNAs also map to the 30-33 nt peak in both serum and plasma. FIG. 16E: tRNAs map to reads in the 30-33 nt peak in serum, but not plasma.

FIG. 17A-FIG. 17E show comparison of small RNA species in human and mouse serum. Human: red; mouse: blue. FIG. 17A: Comparison of read length distributions of small RNAs extracted from human and mouse sera. Length distribution is displayed by percentage of sequencing reads that map to the hg19 human genome or mm10 mouse genome. Reads were mapped with Bowtie according to the end-to-end k-difference policy allowing two mismatches and multiple reportable alignments for each read. FIG. 17B: Comparison of the annotated noncoding small RNAs in human and mouse serum. The x-axis denotes the types of annotated small RNAs: YRNAs, tRNAs, rRNAs, and Other (other noncoding small RNAs including snRNAs and snoRNAs). The y-axis represents the reads that map to the indicated small RNA type as percentage of the total reads sequenced from the human or mouse serum samples. FIG. 17C: YRNAs map to reads in the 27 nt peak in human serum, but are scarce in mouse serum. FIG. 17D: YRNAs are present in the 30-33 nt size range in human, but are scarce in mouse serum. FIG. 17E: tRNAs map to reads in the 30-33 nt peak in mouse, but are scarce in human. In FIG. 17C-FIG. 17E, the x-axis represents the read length in nucleotides. The y-axis represents the percentage of the total number of reads sequenced from the human or mouse serum samples that are of the indicated length.

### DETAILED DESCRIPTION OF THE INVENTION

### I. Definitions

As used herein, the following terms have the meanings ascribed to them unless specified otherwise.

In this disclosure the term “or” is generally employed in its sense including “and/or” unless the content clearly dictates otherwise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a RNA or polypeptide chain. It may include regions preceding and following the non-coding region. It may also include regions preceding and following the coding region (leader and trailer) as well as intervening sequences (introns) between individual coding segments (exons).

The term “nucleotide” covers naturally occurring nucleotides as well as nonnaturally occurring nucleotides. It should be clear to the person skilled in the art that various nucleotides which previously have been considered “non-naturally occurring” have subsequently been found in nature. Thus, “nucleotides” includes not only the known purine and pyrimidine heterocycles-containing molecules, but also heterocyclic analogues and tautomers thereof. Illustrative examples of other types of nucleotides are molecules containing adenine, guanine, thymine, cytosine, uracil, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleotides described in U.S. Pat. No. 5,432,272. The term “nucleotide” is intended to cover every and all of these examples as well as analogues and tautomers thereof. Especially interesting nucleotides are those containing adenine, guanine, thymine, cytosine, and uracil, which are considered as the naturally occurring nucleotides in relation to therapeutic and diagnostic application in humans. Nucleotides include the natural 2′-deoxy and 2′-hydroxyl sugars, e.g., as described in Kornberg and Baker, DNA Replication, 2nd Ed. (Freeman, San Francisco, 1992) as well as their analogs.

In this disclosure the term “isolated” nucleic acid molecule means a nucleic acid molecule that is separated from other nucleic acid molecules that are usually associated with the isolated nucleic acid molecule. Thus, an “isolated” nucleic acid molecule includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated” nucleic acid.

“Purified polynucleotide” or “isolated polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

“Analogs” in reference to nucleotides includes synthetic nucleotides having modified base moieties and/or modified sugar moieties. Such analogs include synthetic nucleotides designed to enhance binding properties, e.g., duplex or triplex stability, specificity, or the like.

“Complementary,” as used herein, refers to the capacity for precise pairing between two nucleotides on one or two oligomeric strands. For example, if a nucleobase at a certain position of an antisense compound is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligomeric compound and the further DNA, RNA, or oligonucleotide molecule are complementary to each other when a sufficient number of complementary positions in each molecule are occupied by nucleotides which can hydrogen bond with each other. Thus, “specifically hybridizable” and “complementary” are terms which are used to indicate a sufficient degree of precise pairing or complementarity over a sufficient number of nucleotides such that stable and specific binding occurs between the oligomeric compound and a target nucleic acid.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. Optionally, the identity exists over a region that is at least about 10, 15, 25 or 50 nucleotides in length, or over the full length of the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “variant” refers to biologically active derivatives of the reference molecule that retain desired activity. In general, the term “variant” refers to molecules (e.g., small non-coding RNAs, microRNAs, tRNAs, YRNAs) having a native sequence and structure with one or more additions, substitutions (generally conservative in nature) and/or deletions, relative to the native molecule, so long as the modifications do not destroy biological activity and which are “substantially homologous” to the reference molecule. In general, the sequences of such variants will have a high degree of sequence homology to the reference sequence, e.g., sequence homology of more than 50%, generally more than 60%-70%, even more particularly 80%-85% or more, such as at least 90%-95% or more, when the two sequences are aligned.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

A “expression vector” or “expression cassette” is capable of transferring nucleic acid sequences to target cells (e.g., viral vectors, non-viral vectors, particulate carriers, and liposomes). Typically, “vector expression cassette” and “expression vector” refer to any nucleic acid construct capable of directing the expression of a nucleic acid of interest and which can transfer nucleic acid sequences to target cells. Thus, the term includes cloning and expression vehicles, as well as viral vectors.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

“Operably linked” refers to an arrangement of elements wherein the components so described are configured so as to perform their usual function. Thus, a given promoter operably linked to a coding sequence is capable of effecting the expression of the coding or non-coding sequence when the proper enzymes are present. Expression is meant to include the transcription of any one or more of transcription of a small non-coding RNA, e.g., microRNA, siRNA, piRNA, snRNA, and lncRNA, antisense nucleic acid, or mRNA from a DNA or RNA template and can further include translation of a protein from an mRNA template. The promoter need not be contiguous with the coding sequence, so long as it functions to direct the expression thereof. Thus, for example, intervening untranslated yet transcribed sequences can be present between the promoter sequence and the coding or non-coding sequence and the promoter sequence can still be considered “operably linked” to the coding or non-coding sequence.

The phrase “differentially expressed” refers to differences in the quantity and/or the frequency of a biomarker present in a sample taken from patients having, for example, cancer caloric restriction, or age-related disease, as compared to a control subject. For example, a biomarker can be a YRNA-derived fragment which is present at an elevated level or at a decreased level in samples of patients with breast cancer compared to samples of control subjects. Alternatively, a biomarker can be a YRNA-derived fragment which is detected at a higher frequency or at a lower frequency in samples of patients with cancer compared to samples of control subjects or control tissues. A biomarker can be differentially present in terms of quantity, frequency or both.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; primates, and transgenic animals.

As used herein, a “biological sample” refers to a sample of tissue or fluid isolated from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

A “polynucleotide hybridization method” as used herein refers to a method for detecting the presence and/or quantity of a pre-determined polynucleotide sequence based on its ability to form Watson-Crick base-pairing, under appropriate hybridization conditions, with a polynucleotide probe of a known sequence. Examples of such hybridization methods include Southern blot, Northern blot, and in situ hybridization.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically a detectable label is attached to a probe or a molecule with defined binding characteristics (e.g., a polypeptide with a known binding specificity or a polynucleotide), so as to allow the presence of the probe (and therefore its binding target) to be readily detectable.

The term “caloric restriction” refers to a diet in which the amount of calories is reduced in comparison to a normal diet without malnutrition. Typically, a caloric restricted diet constitutes about 90% or 85%, often 80%, 75%, 70%, 65%, 60%, 55%, or 50% of a normal diet for a subject. As appreciated by one of skill in the art, a normal diet is determined with respect to factors such as age, sex, height and body frame, and the like.

The term “biomarker of caloric restriction” refers to a nucleic acid sequence that is differentially expressed in caloric-restricted subject. Caloric-restricted biomarkers include those that are up-regulated (i.e., expressed at a higher level) in caloric-restriction, as well as those that are down-regulated (i.e., expressed at a lower level).

The term “up-regulation” means that the ratio of the level of product in treated vs. control is greater than one. Often, the ratio is 1.1, 1.3, 1.5, 2.0 or greater. As appreciated by those in the art, statistical analysis is typically performed to evaluate significance.

The term “down-regulation” as used herein means that the ratio of the level of product in treated vs. control is less than one. Often the ratio is 0.75, 0.5, 0.25 or less. As appreciated by those in the art, statistical analysis is typically performed to evaluate significance.

### II. General Methodology

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

### III. Detailed Description of the Embodiments

### A. Small Non-Coding RNAs

As disclosed above, the small non-coding RNAs used herein refer to 5′ tRNA halves that are derived from specific tRNAs (e.g., as in Tables 3 and 4). For instance, the 5′ tRNA halves having a nucleic acid sequence corresponding to the first 27-35, e.g., 27, 28, 29, 30, 31, 32, 33, 34, 35 nucleic acids of a tRNA gene. The sncRNAs also refer to the YRNA fragments derived from specific YRNAs (e.g., as in Table 5). For instance, the YRNA fragments, having a nucleic acid sequence corresponding to the first 27-35, e.g., 27, 28, 29, 30, 31, 32, 33, 34, 35 nucleic acids of a YRNA gene (or pseudogene).

The 5′ tRNA half can be generated from the specific tRNA from which it is derived. For example, a tRNA can be cleaved by, e.g., an in vitro cleavage reaction, to generate its cognate 5′ tRNA half. Similarly, a YRNA fragment can be produced from its cognate YRNA by cleavage.

The source of the sncRNA can be naturally-occuring or synthetic. In some embodiments, a synthetic sncRNA can have a sequence that is different from a naturally-occurring sncRNA and effectively mimic the naturally-occurring sncRNA. For example, the synthetic sncRNA can have at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater sequence similarity to the naturally-occurring sncRNA.

Synthetic polynucleotides or oligonucleotides can be generated by, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

### B. Generating a Small Non-Coding RNA Expression Constructs

In some embodiments, expression vector that comprise a heterologous promoter and a polynucleotide sequence for a tRNA or YRNA (e.g., as provided in Tables 3-5) is generated and introduced to a host cell (e.g., a eukaryotic cell, a prokaryotic cell, a human cell, and a cell line). Examples of promoters include, but are not limited to, inducible promoters, constitutive promoters, enhancers, and other regulatory elements. In some embodiments, the promoter is an elongation factor 1α (EF1α) promoter, a U6 promoter, or a CMV promoter. In addition to the tRNA or YRNA sequence and the promoter to which it is operably linked, the expression cassette may contain one or more additional components, including, but not limited to regulatory elements such as enhancers. In some embodiments, the sncRNA sequence is optionally associated with a regulatory element that directs the expression of the sncRNA sequence in a target cell.

In some embodiments, the expression vector can replicate and direct expression of a sncRNA in the target cell. Various expression vectors that can be used herein include, but are not limited to, expression vectors that can be used for nucleic acid expression in prokaryotic and/or eukaryotic cells. Non-limiting examples of expression vectors for use in prokaryotic cells include pUC8, pUC9, pBR322 and pBR329 available from BioRad Laboratories, (Richmond, Calif.), pPL and pKK223 available from Pharmacia (Piscataway, N.J.). Non-limiting examples of expression vectors for use in eukaryotic cells include pSVL and pKSV-10 available from Pharmacia; pBPV-1/pML2d (International Biotechnologies, Inc.); pcDNA and pTDT1 (ATCC, #31255); viral vectors based on vaccinia virus, poliovirus, adenovirus, adeno-associated virus, herpes simplex virus, a lentivirus; vectors derived from retroviruses such as Rous Sarcoma Virus, Harvey Sarcoma Virus, avian leukosis virus, human immunodeficiency virus, myeloproliferative sarcoma virus, and mammary tumor virus); and the like. Additional examples of suitable eukaryotic vectors include bovine papilloma virus-based vectors, Epstein-Barr virus-based vectors, SV40, 2-micron circle, pcDNA3.1, pcDNA3.1/GS, pYES2/GS, pMT, p IND, pIND(Sp1), pVgRXR (Invitrogen), and the like, or their derivatives

In some embodiments, the expression vectors disclosed herein can include one or more coding regions that encode a polypeptide (a “marker”) that allows for detection and/or selection of the genetically modified host cell comprising the expression vectors. The marker can be a drug resistance protein such as neomycin phosphotransferase, aminoglycoside phosphotranferase (APH); a toxin; or fluorescence. Various selection systems that are well known in the art can be used herein. The selectable marker can optionally be present on a separate plasmid and introduced by co-transfection.

Skilled artisans will appreciate that any methods, expression vectors, and target cells suitable for adaptation to the expression of a 5′ tRNA or YRNA in target cells can be used herein and can be readily adapted to the specific circumstances.

### C. Quantitating a Small Non-Coding RNA

In certain embodiments, the disclosure relates to methods of analyzing samples for expression of sncRNA or RNA disclosed herein. Typical methods are based on hybridization analysis of polynucleotides, and sequencing of polynucleotides. The most commonly used methods known in the art for the quantification of RNA expression in a sample include northern blotting and in situ hybridization; RNAse protection assays; and reverse transcription polymerase chain reaction (RT-PCR). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In certain embodiments, a sncRNA detection agent such as a complementary nucleotide sequence can be labeled to allow detection in an imaging system, such as a positron emission tomography (PET) scan, single-photon emission computed tomography (SPECT) or a similar type of scan by administering the labeled detection agent to the subject and then scanning the brain of the subject for binding. In those instances the detection agent may be labeled so as to only emit signal if bound to the sncRNA.

Reverse Transcriptase PCR (RT-PCR) may be used to compare sncRNA levels in different sample populations, in normal and disease samples, with or without drug treatment, to characterize patterns of sncRNA levels, to discriminate between closely related sncRNAs, and to analyze RNA structure. This method typically employs isolation of sncRNA from a target sample, e.g., blood, serum, plasma or other bodily fluid.

General methods for nucleic acid (e.g., RNA) extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. RNA may be isolated, for example, by cesium chloride density gradient centrifugation.

RT-PCR can be performed using commercially available equipment, such as the ABI PRISM 7700™ Sequence Detection System™. Differential RNA expression can also be identified, or confirmed using the microarray technique.

In addition, methods of measuring sncRNA include contacting a sample from a subject with a probe, which can be a nucleic acid-containing compound. Such nucleic acid-containing compound can be complementary to at least a portion, including at least 2, at least 3, at least 4, at least 5, at least 6, at least 7, at least 8, at least 9, at least 10, or at least 11 or more nucleic acids of the sncRNA sequence. The probe can also be complementary to at least 50% m at least 60%, at least 70%, at least 80%, at least 90% or at least 95%, at least 98%, or more of the sncRNA sequence. The probe can itself emit a signal or be linked to or bind to a compound that emits a signal, that can be measured, or can be used in a method of measurement such as during a PCR-based technique.

### D. Diagnosing Health Status Using a Small Noncoding RNA

The present invention related to assaying sncRNA (e.g., 5′tRNA halves and YRNA fragments) to determine or monitor an individual's health status, e.g., aging and/or caloric restriction. The present invention also relates to the use of sncRNA biomarkers to detect cancer, e.g., breast cancer. More specifically, the biomarkers of the present invention can be used in diagnostic tests to determine, characterize, qualify, and/or assess cancer status, for example, to diagnose cancer, in an individual, subject or patient.

In some embodiments, the presence or level of one or more 5′ tRNA halves, e.g., 1, 2, 3, 4, 5, 6, 7, 8, 9, 10, 20, 30, 40, 50 or more 5′ tRNA halves, are used to determine a subject's health status. In some cases, the 5′ tRNA halves are selected from those disclosed in Tables 3 and 4 and Dhahbi et al., BMC Genomics, 2013, 14:298, the disclosure of which is herein incorporated by reference in its entirety for all purposes.

In some embodiments, the presence or level of one or more YRNA fragments, e.g., 1, 2, 3, 4, 5, 6, 7, 8, 9, 10, 20, 30, 40, 50 or more YRNA fragment, are used to determine a subject's health status. In some cases, the 5′ YRNA fragment are selected from those disclosed in Table 5 and Dhahbi et al., Physiol Genomics, 2013, 45(21):990-998, the disclosure of which is herein incorporated by reference in its entirety for all purposes.

Detection and quantification of RNA expression can be achieved by any one of a number of methods well known in the art, including those described above. For instance, using the known sequences for the sncRNA biomarkers, specific probes and primers can be designed for use in the detection methods described herein as appropriate.

In some cases, the RNA detection method requires isolation of nucleic acid from a sample, such as a cell or tissue sample. Nucleic acids, including RNA and specifically scnRNAs, can be isolated using any suitable technique known in the art. For example, phenol-based extraction is a common method for isolation of RNA. Phenol-based reagents contain a combination of denaturants and RNase inhibitors for cell and tissue disruption and subsequent separation of RNA from contaminants. Phenol-based isolation procedures can recover RNA species in the 10-200-nucleotide range (e.g., sncRNAs). In addition, extraction procedures such as those using TRIZOL™ or TRI REAGENT™, will purify all RNAs, large and small, and are efficient methods for isolating total RNA from biological samples that contain small non-coding RNAs.

### E. Kits

For use in diagnostic, research and therapeutic applications suggested above, kits are also provided by the invention. In the diagnostic and research applications such kits may include any or all of the following: assay reagents, buffers, hybridization probes and/or primers, control small non-coding RNAs, etc. A therapeutic product may include sterile saline or another pharmaceutically acceptable emulsion and suspension base.

The kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), digital media, and the like. Such media may include addresses to internet sites that provide such instructional materials.

A wide variety of kits and components can be prepared according to the present invention, depending upon the intended user of the kit and the particular needs of the user.

### IV. Examples

The following examples are offered to illustrate, but not to limit the claimed invention.

### Example 1

### 5′ tRNA Halves are Present as Abundant Complexes in Serum, Concentrated in Blood Cells, and Modulated by Aging and Calorie Restriction

Small RNAs complex with proteins to mediate a variety of functions in animals and plants. Some small RNAs, particularly miRNAs, circulate in mammalian blood and may carry out a signaling function by entering target cells and modulating gene expression. The subject of this study is a set of circulating 30-33 nt RNAs that are processed derivatives of the 5′ ends of a small subset of tRNA genes, and closely resemble cellular tRNA derivatives (tRFs, tiRNAs, half-tRNAs, 5′ tRNA halves) previously shown to inhibit translation initiation in response to stress in cultured cells.

In sequencing small RNAs extracted from mouse serum, we identified abundant 5′ tRNA halves derived from a small subset of tRNAs, implying that they are produced by tRNA type-specific biogenesis and/or release. The 5′ tRNA halves are not in exosomes or microvesicles, but circulate as particles of 100-300 kDa. The size of these particles suggest that the 5′ tRNA halves are a component of a macromolecular complex; this is supported by the loss of 5′ tRNA halves from serum or plasma treated with EDTA, a chelating agent, but their retention in plasma anticoagulated with heparin or citrate. A survey of somatic tissues reveals that 5′ tRNA halves are concentrated within blood cells and hematopoietic tissues, but scant in other tissues, suggesting that they may be produced by blood cells. Serum levels of specific subtypes of 5′ tRNA halves change markedly with age, either up or down, and these changes can be prevented by calorie restriction.

We demonstrate that 5′ tRNA halves circulate in the blood in a stable form, most likely as part of a nucleoprotein complex, and their serum levels are subject to regulation by age and calorie restriction. They may be produced by blood cells, but their cellular targets are not yet known. The characteristics of these circulating molecules, and their known function in suppression of translation initiation, suggest that they are a novel form of signaling molecule.

Several classes of small RNAs have been found to mediate biological functions in animals and plants [1-5]. miRNAs, siRNAs, piRNAs, and others are bound by Argonaute proteins, and have the common property of directing protein complexes to nucleic acids with sequence complementarity, where they may cleave or otherwise alter the target [6]. In both plants and animals, some small RNAs are able to travel between tissues within an organism, thus transferring their functions to other cells. In vertebrates, there has been much recent interest in the presence of specific miRNAs in the plasma and serum; there is some evidence that these can be taken up by cells and alter gene expression, and there is also interest in the possibility that they can be markers of specific disease states, including cancer [7-9].

There is also evidence for processing of non-coding RNAs into smaller RNAs, many with as yet poorly understood functions [10, 11]. Many of the non-coding RNAs that appear to undergo processing into smaller RNAs have well studied functions, although their smaller derivatives often do not. In particular, tRNA is processed into shorter forms termed tRNA fragments (tRFs) [12, 13]. The subject of this report is a tRNA fragment created by cleavage of tRNA near the anticodon loop to create a “5′ tRNA half” (the term we will use here). Previous reports have described 5′ tRNA halves as intracellular molecules interacting with components of the translation initiation complex. 5′ tRNA halves have been shown to be induced by the ribonuclease angiogenin in response to stress in cultured cells, to promote assembly of stress granules carrying stalled preinitiation complexes, and to inhibit mRNA translation [14, 15]; little more is known about their function.

We have sequenced small RNAs present in mouse serum; when multiple reportable alignments of the sequencing reads to the mouse genome were allowed, we noted the presence of a class of tRNA-derived 30-33 nt fragments that closely resemble the 5′ tRNA halves previously described in stressed cell cultures. Investigation of these 5′ tRNA halves reveals a novel class of circulating small RNAs whose characteristics, including changes with age that are antagonized by calorie restriction, strongly suggest physiologic regulation and function.

### Results

### Sequencing and Computational Analysis of Small RNAs Circulating in Mouse Serum

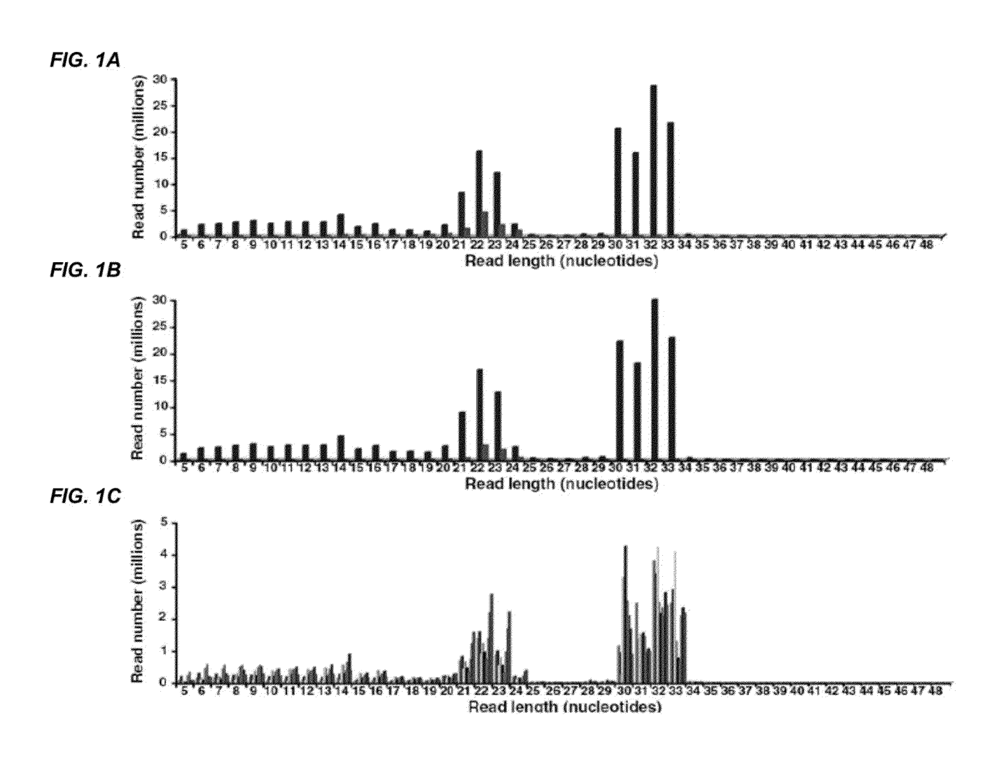

While investigating the effects of aging and calorie restriction (CR) on the profiles of cell-free small RNAs circulating in the bloodstream, we used small RNA-Seq (Illumina reads of 50 nt) to compare the serum levels of small RNAs from young and old control mice, and old mice subjected to CR. A combined total of 196,083,881 pre-processed sequencing reads obtained from 9 different serum samples, were mapped to the mouse genome with bowtie using parameters that align reads according to a policy similar to Maq's default policy [16]. Alignment of the combined 196,083,881 pre-processed sequencing reads generated a dataset of 163,078,230 mapped reads (83.2%), ranging from 5 to 48 nt. The size distribution of the mapped reads revealed an expected peak at 20-24 nt consistent with the size of miRNAs (FIG. 1).

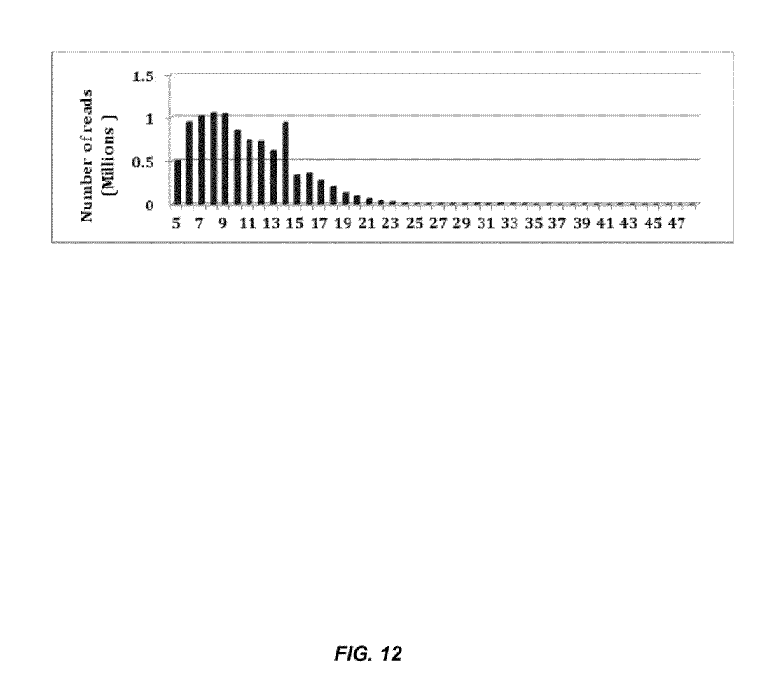

Only if multiple reportable alignments are allowed during bowtie mapping does an unfamiliar second peak emerge at 30-33 nt (FIG. 1A). The 30-33 nt peak persists when the bowtie alignment mode is changed from the Maq's default policy (n option) to the end-to-end k-difference policy (v option), but again disappears when multiple reportable alignments are suppressed (FIG. 1B). The same two-peak pattern was observed when the 9 individual sequenced serum small RNA samples were mapped to the mouse genome (FIG. 1C). Dependence of the 30-33 nt peak on multiple reportable alignments indicates that the reads are encoded by repetitive DNA. Six percent of the 163,078,230 mapped reads, aligned to a group of RepeatMasker classes (DNA, LINE, LTR, Low complexity, RC, SINE, Satellite, and Simple repeat); these reads were mainly <20 nt in size (FIG. 12) and were not considered for further analysis.

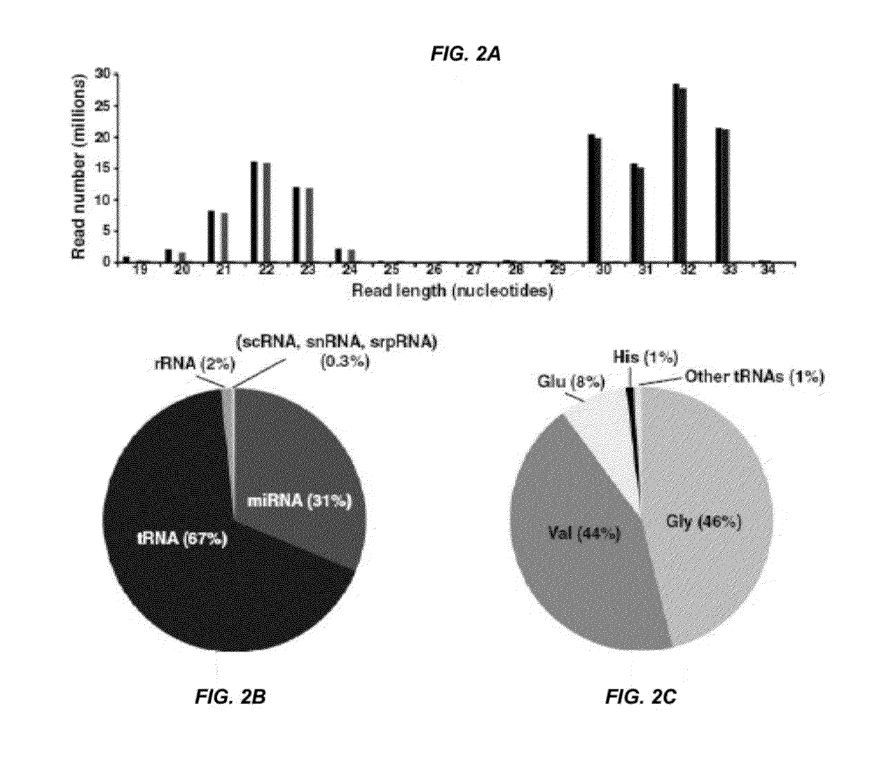

Annotation analysis of the mapped sequencing reads revealed that the 30-33 nt peak consists of reads mapping to tRNA genes (FIG. 2A), which are present in multiple copies in the genome. Reads in the 20-24 nt peak were mostly annotated as miRNAs. Further analysis showed that of the total 163,078,230 reads that mapped to the mouse genome, 128,703,415 (79%) map to sequences encoding small RNAs, of which 67% and 31% were annotated as tRNAs and miRNAs, respectively (FIG. 2B). The remaining <3% of reads mapped to sequences annotated as encoding rRNA and other small RNAs (scRNA, snRNA, srpRNA).

Characterization of Circulating Small RNAs Derived from tRNAs

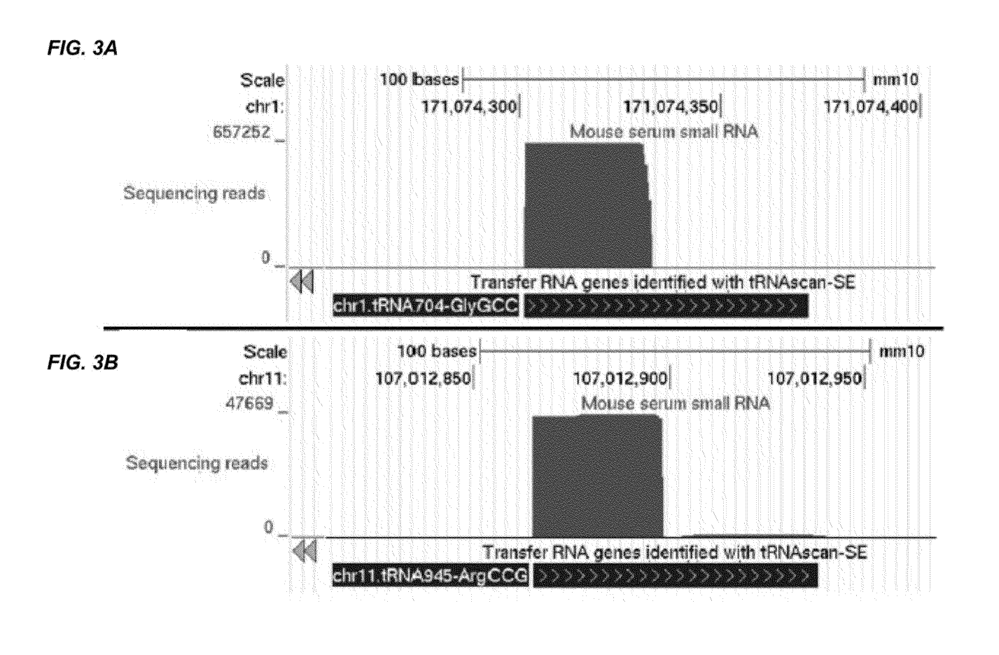

Since the 86,343,437 reads that align to tRNA genes are only 30 to 33 nt, and thus do not represent full length tRNAs, we examined the tRNA end distribution of the reads, and annotated the reads based on their overlap with 5′ or 3′ ends of tRNAs. More than 99% of the tRNA-derived reads align with the 5′ end of a tRNA; this is exemplified in FIG. 3 for two tRNA genes.

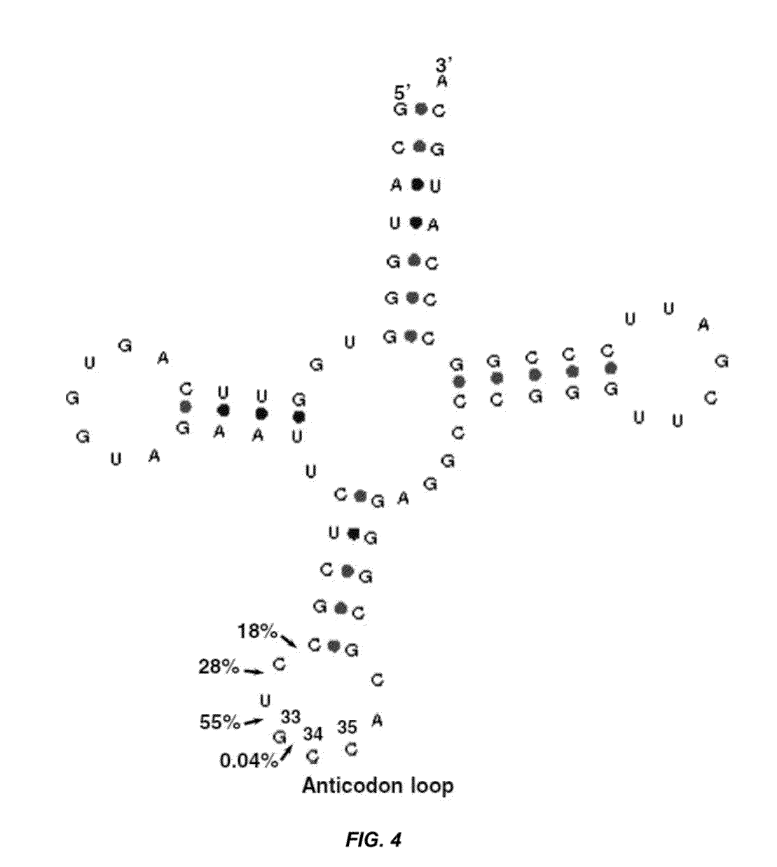

23%, 17%, 35%, and 26% of the sequencing reads that map to tRNAs are 30, 31, 32, and 33 nucleotide in size, respectively (Table 1), indicating that full length tRNAs are cleaved in the anticodon loop at more than one site and at varying rates to generate the 5′ tRNA halves found in serum. As an example, FIG. 4 depicts the size frequency of reads mapping to the 5′ end of the chr1.tRNA704-GlyGCC gene; this indicates that this tRNA is cleaved at different rates and at 4 different sites located upstream of the GCC anticodon in the anticodon loop.

TABLE 1
Total number and percentage of the different sizes of sequencing reads
that map to tRNAs

| Read size in nucleotides | number of reads | Percentage of reads |
|---|---|---|
| 30 | 16649224 | 23% |
| 31 | 12343893 | 17% |
| 32 | 25190160 | 35% |
| 33 | 18724475 | 26% |

It is unlikely that this result is a sequencing artifact: the full length of most tRNAs is 75-90 nt, and the sequencing runs used to generate these data were 50 cycles while the reads occupy a narrow size range of 30-33 nt. This pattern suggests that the tRNA reads were derived from processed fragments of full length tRNAs; the remainder of the tRNA was not significantly detected in the serum small RNA libraries. In support of this conclusion, tRNAs have been shown to undergo cleavage within anticodon loops to produce tRNA-derived stress-induced fragments (tiRNAs) when cultured cells are subjected to stresses such as arsenite, heat shock, or ultraviolet irradiation [17, 18]. Such cleavage of the anticodon loop does not seem to be part of a tRNA degradation process, because the generated 5′ tRNA fragments are stable in the cell. Our findings indicate that tRNA fragments highly similar to tiRNAs are present under normal (unstressed) conditions, and can remain stable even after they are released into the peripheral blood. 5′ but not 3′ tRNA fragments inhibit mRNA translation initiation in cultured cell lines [18].

The individual 5′ tRNA halves present in serum are derived from a small subset of tRNAs (FIG. 2C). The most abundant circulating tRNA halves were derived from the isoacceptors of glycine (46%), valine (44%), glutamine (8%), and histidine (1%), and the remaining amino acids together represented <1%. We contrasted the number of tRNA genes in the Genomic tRNA Database [19], with the relative abundances of the circulating 5′ tRNA halves, and found no correlation (Table 2). For example, the most abundant circulating 5′ tRNA halves were derived from tRNA-Gly, and the copy number of tRNA-Gly gene is 29; on the other hand tRNA-Cys genes, with a copy number of 57, generate <1% of the 5′ tRNA-Cys halves.

TABLE 2
Frequencies of circulating 5′ tRNA halves and the gene copy number
of tRNAs from which the tRNA halves were derived

| | | % of |
|---|---|---|
| tRNA type | gene copy number | circulating tRNA halves |
| Gly | 29 | 46% |
| Val | 23 | 44% |
| Glu | 21 | 8% |
| His | 11 | 1% |
| Others* | 351 | <1% |

*All the remaining tRNAs combined.

This implies a tRNA type-specific biogenesis and/or release of the circulating 5′ tRNA halves.

Presence in Circulating Mouse Blood of Particles Containing Stable Cell-Free 5′ tRNA Halves

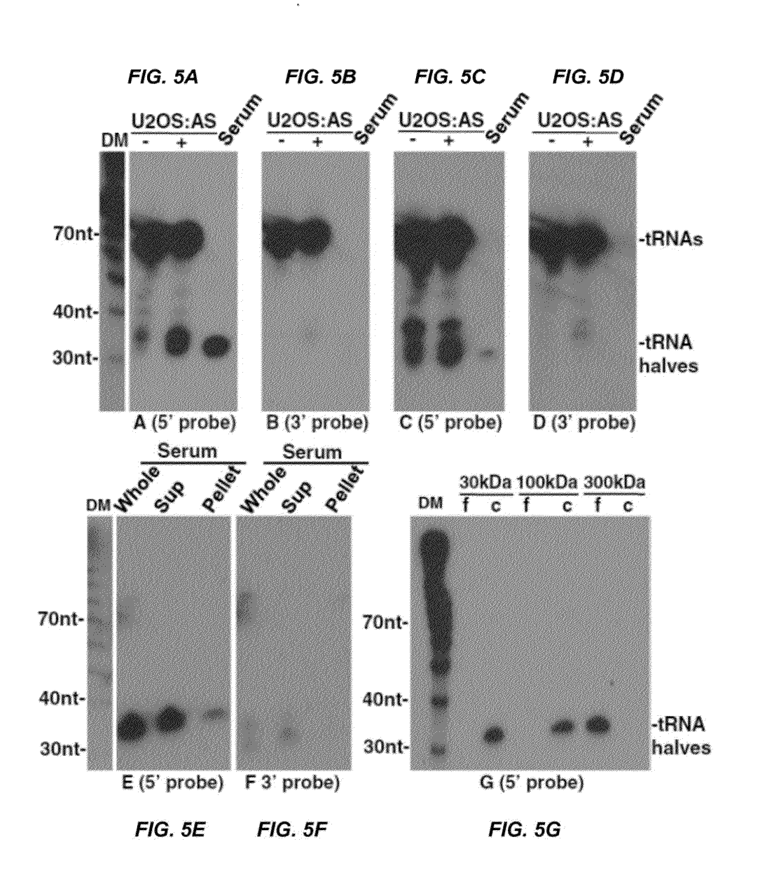

To obtain an independent validation of the sequencing results, we used Northern blotting to analyze small RNAs circulating in the mouse serum. As a positive control for detection of tRNA halves by Northern blotting, we included RNA from U2OS cells cultured in the absence or presence of sodium arsenite, which is known to generate tRNA halves in these cells [18]. We probed RNA from mouse serum with oligonucleotides complementary to 5′ or 3′ ends of specific tRNAs. Probes specific for the 5′ ends of tRNA-Gly-GCC or tRNA-Val-CAC detected a band migrating near the 30 nt RNA marker (FIGS. 5A and C) confirming the presence of stable circulating 5′ tRNA halves. No significant bands migrated with the 30 nt RNA marker when the same Northern blot was probed for the 3′ end of tRNA-Gly-GCC or tRNA-Val-CAC (FIGS. 5B and D).

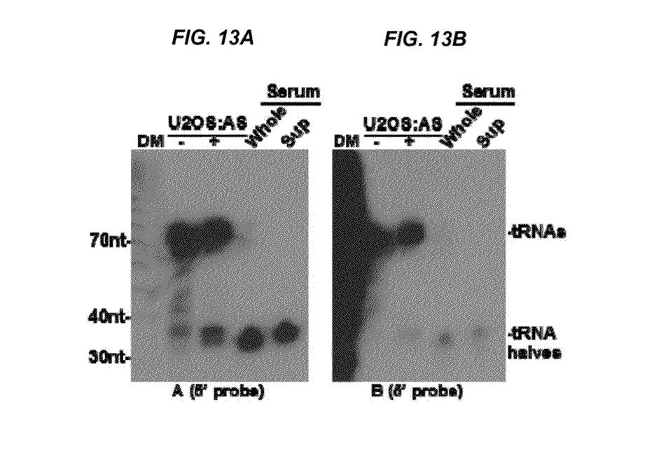

We also probed RNA from mouse serum with a probe complementary to the 5′ end of tRNA-Asn-GTT to confirm the low abundance of circulating tRNA halves derived from tRNAs that were barely detected in the sequencing data. A 5-day exposure to X-ray film showed a very weak signal from tRNA-Asn-GTT probe compared to the strong signal from the tRNA-Gly-GCC probe obtained after a short (25 minute) exposure (FIG. 13). These results are consistent with the sequencing, and inconsistent with a sequencing bias. They imply a tRNA type-specific biogenesis and/or release of the circulating 5′ tRNA halves.

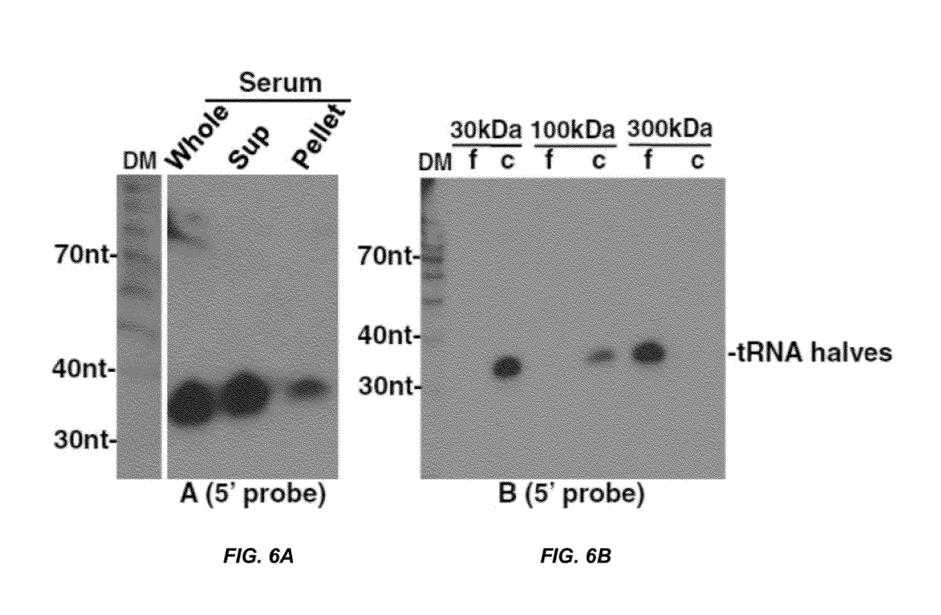

We next asked if the 5′ tRNA halves are contained within circulating exosomes or microvesicles. We Northern blotted RNA extracted from pellet or supernatant after ultracentrifugation of mouse serum at 110,000 g for 2 hours. A probe for the 5′ end of tRNA-Gly-GCC detected an ˜30 nt band present mainly in the supernatant and visible only as a trace in the pellet (FIG. 5E), while a probe for the 3′ end did not detect any significant signal (FIG. 5F). Identical results were obtained for the 5′ end of tRNA-Val-CAC (FIG. 6A). These findings indicate that the 5′tRNA halves are mostly not included in exosomes or microvesicles, which would pellet in these conditions. Similarly, exosome encapsulation is not required for the stability of circulating miRNAs; after pelleting exosomes by ultracentrifugation of plasma, miRNAs were still detected in the supernatant fraction [20, 21].

Because the tRNA halves we observe are stable in circulation but not encapsulated in exosomes, they are most likely complexed to carrying factors (e.g., proteins that protect them from degradation). To determine the size range of the putative complexes carrying the 5′ tRNA halves in the serum, we Northern blotted RNA extracted from concentrate or filtrate fractions after ultrafiltration of mouse serum samples through Vivaspin 2 columns with 30, 100, or 300 kDa MW cut-off. A probe for the 5′ end of tRNA-Gly-GCC detected a ˜30 nt band in the concentrates of 30 and 100 kDa MW cut-off, and in the filtrate of 300 kDa MW cut-off (FIG. 5G). Identical results were obtained for the 5′ end of tRNA-Val-CAC (FIG. 6B).

Thus 5′ tRNA halves circulate as part of 100-300 kDa complexes, while the 5′ tRNA halves themselves are only ˜10 kDa. This is reminiscent of reports that miRNAs can circulate in the bloodstream as components of RNA-protein/lipoprotein complexes. Stable argonaute 2-miRNA complexes that are not part of microvesicles were recovered from plasma and serum, and high-density lipoprotein has been reported to carry and deliver miRNAs to recipient cells [20-22].

5′ tRNA Halves are Concentrated in Hematopoietic and Lymphoid Tissues

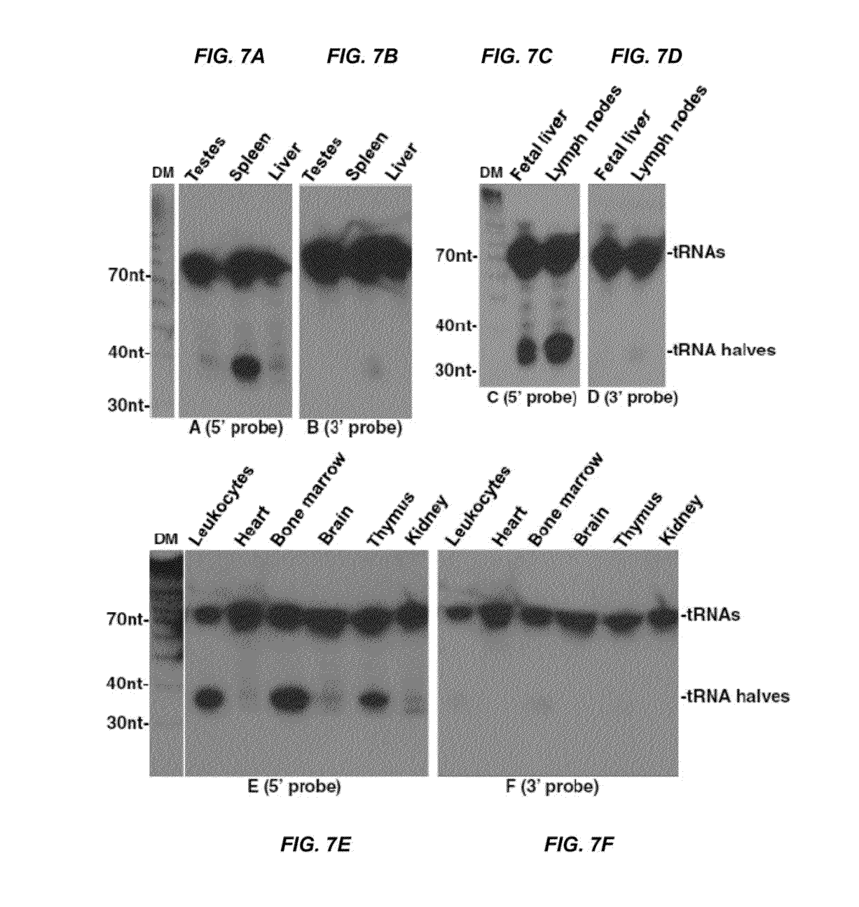

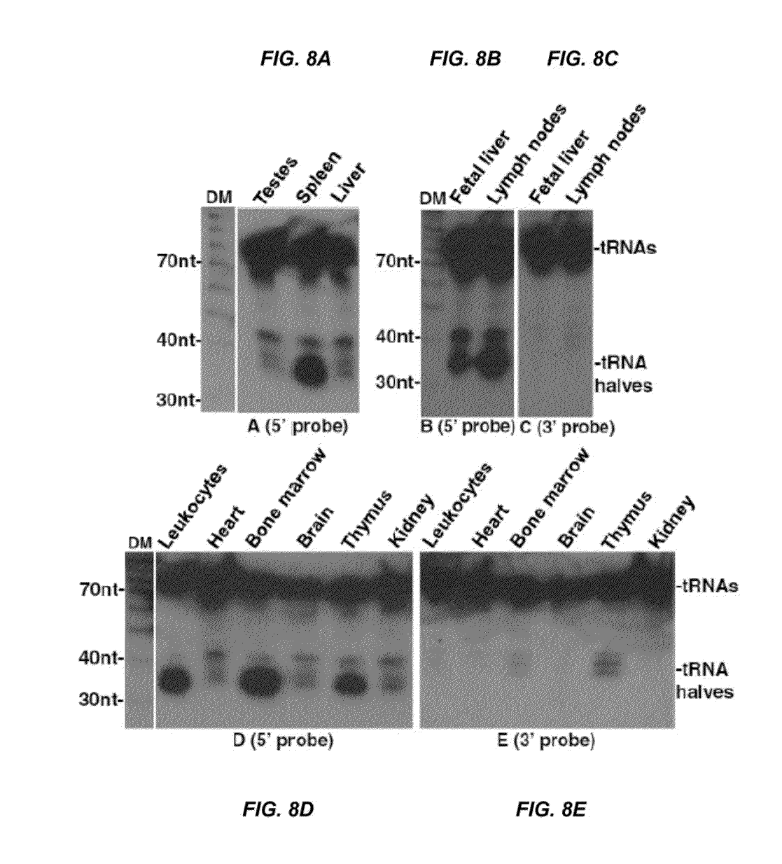

To investigate whether 5′ tRNA halves are present in tissues we extracted total RNA from liver, spleen, and testes, and did Northern blots with probes complementary to 5′ and 3′ ends of tRNAs. We detected tRNA halves with a probe complementary to the 5′ end of tRNA-Gly-GCC in the spleen, but not in the liver and testes; a probe for the 3′ end tRNAs detected only full length tRNAs in all 3 tissues (FIG. 7A-B). This prompted us to explore the possibility that 5′ tRNA halves are present specifically in hematopoietic tissues. Northern blotting of several mouse tissues confirmed that 5′ tRNA halves are present in hematopoietic and lymphoid tissues including spleen, lymph nodes, fetal liver, leukocytes, bone marrow, and thymus, but almost absent in non-immune tissues including testes, liver, heart, brain, and kidney (FIG. 7); the presence of 3′ tRNA halves was not significant in any tissue. This finding is consistent with a previous report [23], in which tRNA halves were unexpectedly detected on cloning of microRNAs from human fetal liver, which is the main hematopoietic organ during fetal development. Identical results were obtained when the same Northern blots were probed for the 5′ and 3′ ends of tRNA-Val-CAC (FIG. 8).

More extensive studies will establish if 5′ tRNA halves are concentrated in particular blood cell types, although the very high levels in lymph nodes point to lymphocytes as one such type. The evidence does not establish whether the 5′ tRNA halves are concentrated in hematopoietic cells because they are produced there, or because they are preferentially taken up from the blood: neither the origin nor the destinations of the 5′ tRNA halves is certain. The low levels of 5′ tRNA halves present in non-hematopoietic tissues may indicate low levels in those tissues, but they may also be derived from residual blood cells in those tissues.

A Chelating Agent Destabilizes Circulating 5′ tRNA Halves

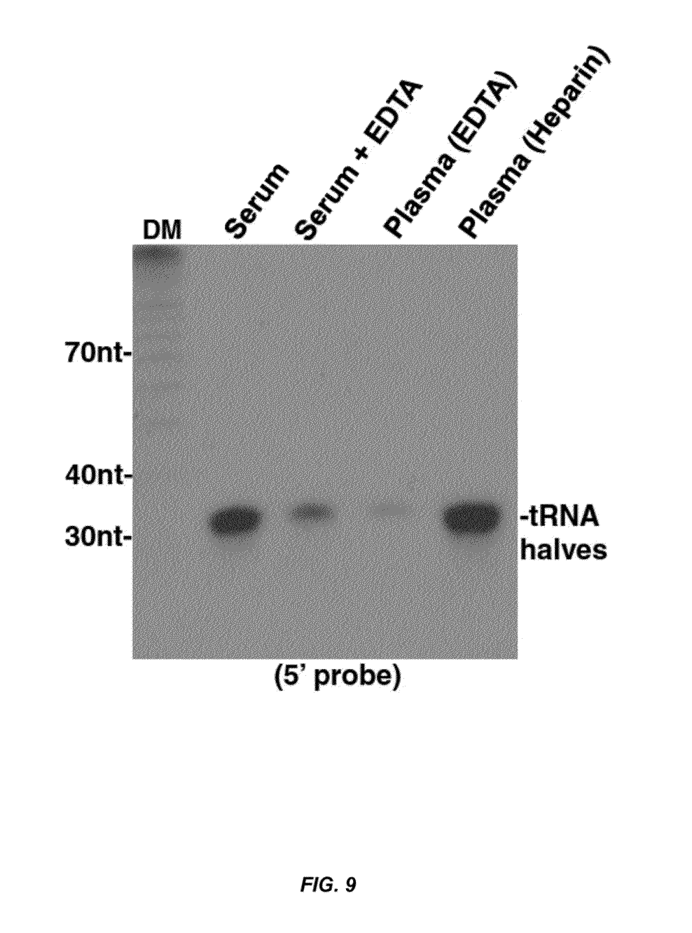

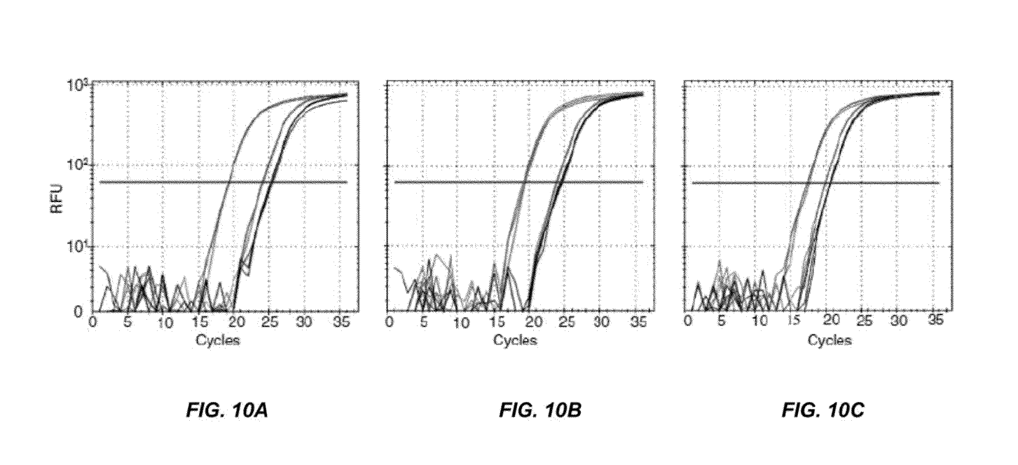

Because clotting has the potential to release particles that are not present in circulating blood, we asked if 5′ tRNA halves circulating in the mouse serum are also present in mouse plasma. Northern blotting with a 5′ tRNA half probe gave a very weak band in a plasma sample when compared to the band derived from an equal volume of serum from the same mouse (FIG. 9). The lack of 5′ tRNA halves in plasma is not due to a global loss of small RNAs during preparation of the plasma, which was anticoagulated with EDTA. We used qPCR to assess the integrity of two circulating miRNAs in mouse serum, serum treated with EDTA, and plasma collected with EDTA. As shown by amplification in all three specimens (FIG. 10), EDTA does not affect these circulating miRNAs.

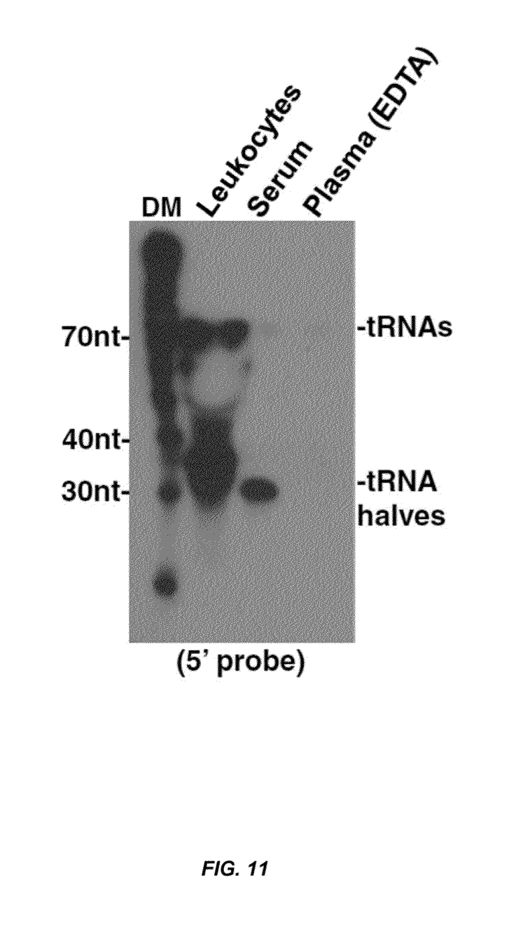

This result could suggest that 5′ tRNA halves are an artifact of blood clotting, but could also be an effect of EDTA, a chelating agent that depletes ions required for clotting. To assess the effects of EDTA on 5′ tRNA halves, we used Northern blotting to analyze a sample of serum that was incubated with EDTA for 15 min before RNA extraction. We also analyzed a sample of plasma extracted from blood collected with heparin, a nonchelating anticoagulant. This analysis showed that treatment of serum with EDTA significantly decreased the signal corresponding the 5′ tRNA halves, while 5′ tRNA halves are abundant in heparinized plasma (FIG. 9). The same results were obtained with RNAs from human plasma collected on EDTA and from serum (FIG. 11). These findings suggest that chelation of ions by EDTA destabilizes the complexes carrying 5′ tRNA halves, exposing the RNA to ribonucleases which are abundant in plasma.

Calorie Restriction Offsets Age-Associated Changes in Levels of Specific Circulating 5′ tRNA Halves

Calorie restriction (CR) can delay, prevent, or reverse many age-associated changes in physiologic parameters. We used aging and CR as model physiologic states to explore the possibility that they are associated with changes in the levels of circulating 5′ tRNA halves. We performed pairwise comparisons between young and old control groups to measure the differential abundance in circulating 5′ tRNA halves associated with old age, and between old control and old CR groups to determine whether CR has an effect on any age-associated changes.

This analysis revealed that aging is associated with alterations, either increase or decrease, in the circulating levels of 5′ tRNA halves derived from specific tRNA isoacceptors (Table 3). Notably, CR mitigated most of these age-related changes (Table 3), although it did not completely prevent them. CR has been shown to oppose the molecular and biological markers of aging including alterations in gene expression [24]. A causal relationship between circulating 5′ tRNA halves and the manifestations of aging is not established by this study, but it does indicate that levels are regulated in an age-associated fashion.

TABLE 3
Age-associated changes in the levels of mouse circulating 5′ tRNA halves and the
effects of CR on the age-associated changes

| | Young | Old | | Age | | CR | |
|---|---|---|---|---|---|---|---|
| tRNA* | control † | control† | Old CR† | FC‡ | p-value | FC‡ | p-value |
| His-GTG | | | | | | | |
| chr4:82619623-82619694 | 797 | 2988 | 1554 | 3.8 | 3.1E−11 | −2 | 6.8E−04 |
| chr2:122377363-122377434 | 798 | 2994 | 1549 | 3.8 | 3.7E−11 | −2 | 6.4E−04 |
| chr3:96452495-96452566 | 307 | 1140 | 590 | 3.8 | 3.8E−11 | −2 | 6.1E−04 |
| chr2:122375494-122375565 | 808 | 2990 | 1533 | 3.8 | 3.9E−11 | −2 | 5.0E−04 |
| chr2:122377968-122378039 | 309 | 1163 | 600 | 3.8 | 4.2E−11 | −2 | 6.6E−04 |
| chr3:96458070-96458141 | 802 | 2993 | 1523 | 3.8 | 4.3E−11 | −2 | 4.8E−04 |
| chr3:96500366-96500437 | 796 | 2954 | 1524 | 3.8 | 4.5E−11 | −2 | 5.9E−04 |
| chr3:96410069-96410140 | 301 | 1148 | 590 | 3.9 | 2.0E−11 | −2 | 5.5E−04 |
| Arg-CCG | | | | | | | |
| chr11: 107012866-107012938 | 1243 | 256 | 302 | −5 | 9.9E−12 | 1.2 | 4.7E−01 |
| Cys-GCA | | | | | | | |
| chr11:97798906-97798977 | 933 | 370 | 688 | −2.6 | 2.5E−06 | 1.8 | 2.4E−03 |
| chr11:97988246-97988317 | 928 | 376 | 700 | −2.5 | 3.9E−06 | 1.8 | 2.2E−03 |
| chr11:97988923-97988994 | 930 | 360 | 684 | −2.6 | 1.3E−06 | 1.9 | 1.5E−03 |
| Gly-GCC | | | | | | | |
| chr1:171074302-171074372 | 16868 | 3739 | 3807 | −4.5 | 3.5E−14 | −1 | 9.6E−01 |
| chr1:171066631-171066701 | 16820 | 3730 | 3790 | −4.5 | 4.3E−14 | −1 | 9.6E−01 |
| chr1:171081876-171081946 | 16779 | 3725 | 3788 | −4.4 | 5.3E−14 | −1 | 9.6E−01 |
| Lys-CTT | | | | | | | |
| chr17:23533962-23534034 | 4175 | 1939 | 3286 | −2.2 | 8.8E−06 | 1.7 | 3.7E−03 |
| chr3:96428235-96428307 | 4215 | 1964 | 3353 | −2.2 | 1.0E−05 | 1.7 | 3.3E−03 |
| chr17:23547360-23547432 | 4098 | 1923 | 3272 | −2.2 | 1.1E−05 | 1.7 | 3.4E−03 |
| chr17:23535332-23535404 | 14085 | 6569 | 11132 | −2.2 | 1.2E−05 | 1.7 | 4.1E−03 |
| chr13:23436340-23436412 | 4181 | 1962 | 3321 | −2.2 | 1.2E−05 | 1.7 | 3.9E−03 |
| chr3:96499512-96499584 | 13865 | 6507 | 11017 | −2.2 | 1.3E−05 | 1.7 | 3.9E−03 |
| chr11:48833883-48833955 | 13905 | 6539 | 11051 | −2.2 | 1.4E−05 | 1.7 | 4.1E−03 |
| Val-AAC | | | | | | | |
| chr13:23401073-23401145 | 1247 | 451 | 814 | −2.8 | 3.3E−07 | 1.8 | 4.1E−03 |
| chr13:23413248-23413320 | 1246 | 467 | 836 | −2.7 | 5.4E−07 | 1.8 | 4.0E−03 |

*tRNA isoacceptor identity with corresponding genomic positions of the tRNA genes in the mouse mm10 genome.
†Average tRNA read count for the indicated experimental group reported as counts per million (cpm) reads in the sequenced library from the indicated experimental group.
‡Fold change calculated by EdgeR from pairwise comparisons between the young and old control groups for the age effect, or between the old control and old CR groups for the CR effect.

### Conclusions

Deep sequencing of small RNAs extracted from mouse serum identifies a population of tRNA-derived molecules, termed 5′ tRNA halves, previously described only as stress-induced inhibitors of translation initiation in cultured cells. 5′ tRNA halves are more abundant than miRNAs in mouse serum, and are derived from distinct subset of tRNAs by cleavage near the anticodon loop; the 3′ portion of the tRNA molecule is present in serum only in trace quantities. Ultracentrifugation and size fractionation establish that the 5′ tRNA halves circulate as part of a larger complex, but are not contained in exosomes or microvesicles; their sensitivity to the chelating agent EDTA provides further evidence that they exist as circulating nucleoprotein complexes. They are concentrated in hematopoietic and lymphoid tissues, and present in other tissues at very low levels that may reflect residual blood cells. The origin of the serum particles, and their destinations, are uncertain; however their concentration in blood cells suggest that they may be produced by these cells. Levels of serum 5′ tRNA halves are distinctly changed in aged mice, and calorie restriction inhibits these changes, indicating that they are subject to physiologic regulation. Taken together with the extant evidence that 5′ tRNA halves can regulate mRNA translation, the characteristics of the circulating 5′ tRNA halves we have discovered suggest that they function as signaling molecules with as yet unknown physiologic roles.

To date, the only known function of 5′ tRNA halves is inhibition of translation in cultured cells subjected to a variety of stressors; transfection of 5′ tRNA halves inhibits global translation in U2OS cells [14, 18]. [14, 18]. A study published while this paper was in preparation reported induction of 5′ tRNA halves in human airway epithelial cells upon infection with respiratory syncytial virus (RSV). Induction involves cleavage at the tRNA anticodon loop by angiogenin, and at least one type, the 5′ tRNA-Glu-CTC half, promotes RSV replication [25]. Our findings indicate that 5′ tRNA halves function on an organismal rather than merely a cellular level. Furthermore they are likely to function in a context much broader than cellular stress or infection: we find 5′ tRNA halves in unstressed conditions. Changes in their expression (either increased or decreased) with age are also consistent with a broader physiologic role, and it is particularly interesting that these changes are partially mitigated by calorie restriction.

The most extensively studied cellular tRNA halves are generated under stress conditions by angiogenin, which cleaves mature tRNAs within the anticodon loops [26]. The stress-induced tRNA halves target the translation initiation machinery to reprogram protein translation in order to promote cell survival during stress [14, 26]. Pull-down and mass spectrometry analyses of RNA-protein complexes have identified several cellular proteins (YB-1, FXR-1, and PABP1) bound to intracellular 5′ tRNA halves [14]. The nature of the proteins and/or other factors that bind and stabilize the extracellular form of 5′ tRNAs halves has yet to be elucidated. Understanding of the origin, composition, and destinations of these complexes will provide insights into their role in organismal physiology.

### Materials and Methods

### Serum Collection, RNA Isolation, and Small RNA Library Construction

Male mice of the long-lived B6C3F1 strain were fed either control or calorie-restricted (CR) diet (40% fewer calories than the control). Three mice were studied from each of three groups: young (7-month) and old (27-month) mice fed the control diet, and old (27-month) mice fed the CR diet. Total RNA including small RNA was isolated from each serum sample with miRNeasy kit (Qiagen) and used to construct indexed sequencing libraries with the Illumina TruSeq Small RNA Sample Prep Kit. The libraries were pooled and sequenced on an Illumina HiSeq 2000 instrument to generate 50 base reads.

Mice and diets. One-month-old male mice of the long-lived B6C3F1 strain were purchased from Harlan (Indianapolis, Ind.). One week after arrival, mice were individually housed and randomly assigned to one of two groups, control or calorie restricted (CR). Control mice were fed 93 kcal/wk of a defined control diet (AIN-93M, diet no. F05312, BIO-SERV). CR mice were fed 52.2 kcal/wk of a defined CR diet (AIN-93M 40% Restricted, diet no. F05314, BIO-SERV). The CR mice consumed ˜40% fewer calories than the control group. The CR diet was enriched so that the CR mice consumed approximately the same amount of protein, vitamins, and minerals per gram of body weight as the control mice. All mice had free access to water. Mice were maintained at 20-24° C. and 50-60% humidity with lights on from 0600 to 1800 h. Sentinel mice were kept in the same room as the experimental mice, and serum samples were screened every 6 months for titers against 11 common pathogens. No positive titers were found during these studies. At 27-months of age, mice were euthanized, and blood was collected through cardiac puncture and processed immediately. A group of control mice were euthanized at 7 months of age and used as a young control group. Each group consisted of 3 mice. The Institutional Animal Care and Use Committee of the University of California, Riverside, approved animal protocols.

RNA isolation, and small RNA library construction. Immediately after collection, blood was transferred to BD Microtainer tubes (Becton, Dickinson and Company), incubated for 30 min at room temperature to allow blood clotting, and centrifuged at 5,000 g for 10 min. The serum supernatant was transferred to new tubes, centrifuged at 16,000 g for 15 min to remove any residual cells and cell-debris, and stored at −80° C. before use. Isolation of total RNA including small RNA was performed with miRNeasy kit (Qiagen) according to the manufacturer's protocol with the exceptions of mixing 2 mL of Qiazol reagent with 0.4 mL serum, loading the entire aqueous phase onto a single column from the MinElute Cleanup Kit (Qiagen), and eluting the RNA in 20 μL of RNase-free water.

One fourth (5 μL) of the RNA isolated from each serum sample was used to construct sequencing libraries with the Illumina TruSeq Small RNA Sample Prep Kit, following the manufacturer's protocol. Briefly, 3′ and 5′ adapters were sequentially ligated to small RNA molecules and the obtained ligation products were subjected to a reverse transcription reaction to create single stranded cDNA. To selectively enrich those fragments that have adapter molecules on both ends, the cDNA was amplified with 15 PCR cycles using a common primer and a primer containing an index tag; this allows multiplexing and sequencing different samples in a single lane of a flowcell. The amplified cDNA constructs were gel purified, and validated by checking the size, purity, and concentration of the amplicons on the Agilent Bioanalyzer High Sensitivity DNA chip. The libraries were pooled in equimolar amounts, and sequenced on an Illumina HiSeq 2000 instrument to generate 50 base reads. Image deconvolution and quality values calculation were performed using the modules of the Illumina pipeline.

RNA extraction from mouse tissues, stressed U2OS cells, fractionated mouse serum and plasma for Northern blot analysis. For stress induction, U2OS cells were cultured in McCoy's 5A Medium supplemented with 10% fetal calf serum and 1% of penicillin/streptomycin, and treated with 500 μM of sodium arsenite (Sigma) for 2 hours before RNA extraction. Tissues and sera were collected from one-year-old mice fed control diet. Tissues were flash frozen in liquid nitrogen. Serum samples were centrifuged at 110,000 g for 2 hrs, and supernatant and pellet fractions were separated. Samples of 0.2 ml serum mixed with 1.8 ml PBS were subjected to ultrafiltration through Vivaspin 2 columns (GE Healthcare) with 30, 100, or 300 kDa MW cut-off, and concentrate and filtrate fractions were collected. All samples were stored at −80° C. before RNA extraction. For plasma preparation, mouse blood samples were mixed with 0.5 M EDTA (10 μl/ml) or sodium heparin (5.5 mg/ml) and centrifuged at 10,000 g for 10 min. The plasma supernatant was transferred to new tubes, centrifuged at 16,000 g for 15 min to remove any residual cells and cell-debris, and stored at −80° C. before use. Total RNA including small RNA was isolated from tissue samples, cell pellets or serum fractions with miRNeasy kit (Qiagen).

Collection of human blood and RNA extraction from serum and plasma. Human blood samples were collected with Institutional Review Board approval after obtaining informed consent. Blood was collected from one young adult male in BD Vacutainer Venous Blood Collection Tubes (BD Diagnostics): K2 EDTA Spray-Dried (BD-366643) or Spray-Coated Sodium Heparin (BD367874). Blood was transferred to Leucosep Centrifuge Tubes (Grenier Bio One #227290P) and centrifuged at 800 g for 15 min at room temperature. The plasma supernatant was transferred to fresh tubes, centrifuged at 16,000 g for 15 min to remove any residual cells and cell-debris, and stored at −80° C. before use. Total RNA including small RNA was isolated from plasma or serum with miRNeasy kit (Qiagen).

Preparation of leukocytes from mouse and human blood and RNA extraction. Blood was collected on EDTA, centrifuged at 1000 g for 15 minutes to separate the plasma and blood cells. The buffy coat was collected, incubated in erythrocyte lysis buffer (Qiagen), and washed with PBS. Leukocyte pellets were flash frozen in liquid nitrogen, and stored at −80° C. before use. Total RNA including small RNA was isolated from leukocytes pellets with miRNeasy kit (Qiagen).

### Mapping and Annotation of Sequencing Reads

Sequencing reads were pre-processed with FASTX-Toolkit (hannonlab.cshl.edu) to trim the adaptor sequences, and discard low quality reads. The obtained clean reads were mapped to the mouse reference genome (GRCm38/mm10) with bowtie version 0.12.8 [16] using different combinations of alignment and reporting options. We used the option “−n 0-114” to align the sequencing reads according to a policy similar to Maq's default policy and requiring no mismatches in the first 14 bases (the high-quality end of the read). In addition, this mode of alignment was combined with options that define which and how many alignments should be reported; the option “−k 1-best” instructed bowtie to report only the best alignment if more than one valid alignment exists, while the option “−m 1” instructed bowtie to refrain from reporting any alignments for reads having multiple reportable alignments. The “−k 1-best” and “−m 1” modes of alignment reporting were also used in combination with the end-to-end k-difference (−v) alignment mode. Varying the alignment and reporting modes allowed the differential detection of two predominant peak sizes of sequencing reads as described in the results section.

Annotation analysis of the mapped sequencing reads was performed with bedtools [27] using the following databases: the Genomic tRNA Database [19] (gtrnadb.ucsc.edu), miRBase 18 (mirbase.org), and rRNA, snRNA, scRNA, and srpRNA which were extracted from the RepeatMasker track (genome.ucsc.edu; mm10).

Analysis of Differentially Abundant Circulating tRNA Halves

The bowtie alignment files generated above from the young and old control and old CR serum sequencing samples were analyzed with bedtools [27] to obtain the coverage of the tRNA genes included in the Genomic tRNA Database [19] (gtrnadb.ucsc.edu), and to determine the read count for each tRNA in the database. The tRNA read counts were further analyzed with the Bioconductor package edgeR [28] to detect the changes in the levels of circulating 5′ tRNA halves in the different experimental groups. The algorithm of edgeR fits a negative binomial model to the count data, estimates dispersion, and measures differences using the generalized linear model likelihood ratio test which is recommended for experiments with multiple factors, such as the simultaneous analysis of age and diet in our study. The fitted count data was analyzed by performing pairwise comparisons between the different experimental groups: young and old control groups were compared to measure the differential abundance in circulating 5′ tRNA halves associated with old age; old control and old CR groups were compared to determine whether CR has an effect on any age-associated changes. The results were further filtered to keep only 5′ tRNA halves that achieved a minimum of 500 counts per million (cpm) in at least one of the 3 experimental groups.

### Northern Blot Assays

RNAs analyzed with Northern blots were extracted from normal or sodium arsenite-treated U2OS and from a variety of tissues and sera harvested from one-year-old mice fed control diet. Before RNA extraction, some serum samples were centrifuged at 110,000×g for 2 hrs, and supernatant and pellet fractions were separated, or were separated into concentrate and filtrate fractions by ultrafiltration through Vivaspin 2 columns with 30, 100, or 300 kDa MW cut-off. RNAs were separated on 15% denaturing polyacrylamide gels, transferred and fixed to a membrane by chemical cross-linking [29], and hybridized with probes complementary to 5′ and 3′ ends of tRNAs.

RNAs extracted from tissue samples, cell pellets or serum fractions as described above were separated on 15% polyacrylamide Criterion TBE-Urea gels (Bio-Rad), transferred to a Hybond NX membrane (GE life sciences), and fixed to the membrane by chemical cross-linking (1). Blots were hybridized overnight at 42° C. in ULTRAhyb-Oligo Buffer (Invitrogen) with the following ³²P-5′-end labeled oligonucleotide probes against the 5′ end of tRNA-Gly-GCC (5′-GGCGAGAATTCTACCACTGAACCACCAA; SEQ ID NO:3), the 3′ end of tRNA-Gly-GCC (5′-TGCATTGGCCGGGAACCGAACCCGGGCCTCCCGCG; SEQ ID NO:4), the 5′ end of tRNA-Val-CAC (5′-AGGCGAACGTGATAACCACTACACTACGGA; SEQ ID NO:5), or the 3′ end of tRNA-Val-CAC (5′-TGTTTCCGCCCGGTTTCGAACCGGGGACCTTTCGCG; SEQ ID NO:6), or the 5′ end of tRNA-Asn-GTT (5′-CGAACGCGCTAACCGATTGCGCCACAGA; SEQ ID NO:7). Membranes were washed twice with 2×SSC, 0.1% SDS solution for 30 minutes, and exposed to X-ray films for detection of signals

Real Time Quantitative PCR (qPCR)

For qPCR assays, 10 fmoles of the synthetic C. elegans cel-miR-39 (Qiagen #MSY0000010) were spiked into 0.2 ml of serum or plasma before RNA extraction to account for variations during RNA extraction, cDNA synthesis, and real-time PCR. One fourth of total RNA extracted from 0.2 ml serum or plasma was reverse transcribed using the miScript Reverse Transcription Kit (Qiagen) according to the manufacturer's protocol. The obtained reverse transcription product was amplified using the following Qiagen reagents: SYBR Green PCR Master Mix, Universal Primer, and miScript Primer Assays for miR-16, miR-24, and miR-Cel-39. Real-time qPCR was carried out on a Bio-Rad CFX96 thermocycler.

### REFERENCES

1. Okamura K: Diversity of animal small RNA pathways and their biological utility. Wiley interdisciplinary reviews RNA 2012, 3(3):351-368.
2. Wery M, Kwapisz M, Morillon A: Noncoding RNAs in gene regulation. Wiley interdisciplinary reviews Systems biology and medicine 2011, 3(6):728-738.
3. Zhang C: Novel functions for small RNA molecules. Current opinion in molecular therapeutics 2009, 11(6):641-651.
4. Zhang S, Sun L, Kragler F: The phloem-delivered RNA pool contains small noncoding RNAs and interferes with translation. Plant physiology 2009, 150(1):378-387.
5. Esteller M: Non-coding RNAs in human disease. Nature reviews Genetics 2011, 12(12):861-874.
6. Joshua-Tor L, Hannon G J: Ancestral roles of small RNAs: an Ago-centric perspective. Cold Spring Harbor perspectives in biology 2011, 3(10):a003772.
7. Allegra A, Alonci A, Campo S, Penna G, Petrungaro A, Gerace D, Musolino C: Circulating microRNAs: New biomarkers in diagnosis, prognosis and treatment of cancer (Review). International journal of oncology 2012, 41(6):1897-1912.
8. Etheridge A, Lee I, Hood L, Galas D, Wang K: Extracellular microRNA: a new source of biomarkers. Mutation research 2011, 717(1-2):85-90.
9. Zen K, Zhang C Y: Circulating microRNAs: a novel class of biomarkers to diagnose and monitor human cancers. Medicinal research reviews 2012, 32(2):326-348.
10. Rother S, Meister G: Small RNAs derived from longer non-coding RNAs. Biochimie 2011, 93(11):1905-1915.
11. Tuck A C, Tollervey D: RNA in pieces. Trends in genetics: TIG 2011, 27(10):422-432.
12. Sobala A, Hutvagner G: Transfer RNA-derived fragments: origins, processing, and functions. Wiley interdisciplinary reviews RNA 2011, 2(6):853-862.
13. Lee Y S, Shibata Y, Malhotra A, Dutta A: A novel class of small RNAs: tRNA-derived RNA fragments (tRFs). Genes & development 2009, 23(22):2639-2649.
14. Ivanov P, Emara M M, Villen J, Gygi S P, Anderson P: Angiogenin-induced tRNA fragments inhibit translation initiation. Molecular cell 2011, 43(4):613-623.
15. Saikia M, Krokowski D, Guan B J, Ivanov P, Parisien M, Hu G F, Anderson P, Pan T, Hatzoglou M: Genome-wide identification and quantitative analysis of cleaved tRNA fragments induced by cellular stress. The Journal of biological chemistry 2012.
16. Langmead B, Trapnell C, Pop M, Salzberg S L: Ultrafast and memory-efficient alignment of short DNA sequences to the human genome. Genome biology 2009, 10(3):R25.
17. Thompson D M, Lu C, Green P J, Parker R: tRNA cleavage is a conserved response to oxidative stress in eukaryotes. RNA 2008, 14(10):2095-2103.
18. Yamasaki S, Ivanov P, Hu G F, Anderson P: Angiogenin cleaves tRNA and promotes stress-induced translational repression. The Journal of cell biology 2009, 185(1):35-42.
19. Chan P P, Lowe T M: GtRNAdb: a database of transfer RNA genes detected in genomic sequence. Nucleic acids research 2009, 37(Database issue):D93-97.
20. Arroyo J D, Chevillet J R, Kroh E M, Ruf I K, Pritchard C C, Gibson D F, Mitchell P S, Bennett C F, Pogosova-Agadjanyan E L, Stirewalt D L et al: Argonaute2 complexes carry a population of circulating microRNAs independent of vesicles in human plasma. In: Proceedings of the National Academy of Sciences of the United States of America. vol. 108, 2011/03/09 edn; 2011: 5003-5008.
21. Turchinovich A, Burwinkel B: Distinct AGO1 and AGO2 associated miRNA profiles in human cells and blood plasma. RNA biology 2012, 9(8).
22. Vickers K C, Palmisano B T, Shoucri B M, Shamburek R D, Remaley A T: MicroRNAs are transported in plasma and delivered to recipient cells by high-density lipoproteins. Nature cell biology 2011, 13(4):423-433.
23. Fu H, Feng J, Liu Q, Sun F, Tie Y, Zhu J, Xing R, Sun Z, Zheng X: Stress induces tRNA cleavage by angiogenin in mammalian cells. FEBS letters 2009, 583(2):437-442.
24. Spindler S R, Dhahbi J M: Conserved and tissue-specific genic and physiologic responses to caloric restriction and altered IGFI signaling in mitotic and postmitotic tissues. Annual review of nutrition 2007, 27:193-217.
25. Wang Q, Lee I, Ren J, Ajay S S, Lee Y S, Bao X: Identification and Functional Characterization of tRNA-derived RNA Fragments (tRFs) in Respiratory Syncytial Virus Infection. Molecular therapy: the journal of the American Society of Gene Therapy 2012.
26. Li S, Hu G F: Emerging role of angiogenin in stress response and cell survival under adverse conditions. Journal of cellular physiology 2012, 227(7):2822-2826.
27. Quinlan A R, Hall I M: BEDTools: a flexible suite of utilities for comparing genomic features. Bioinformatics 2010, 26(6):841-842.
28. Robinson M D, McCarthy D J, Smyth G K: edgeR: a Bioconductor package for differential expression analysis of digital gene expression data. Bioinformatics 2010, 26(1):139-140.
29. Pall G S, Hamilton A J: Improved northern blot method for enhanced detection of small RNA. Nature protocols 2008, 3(6):1077-1084.

### Example 2

### 5′ YRNA Fragments Derived by Processing of Transcripts from Specific YRNA Genes and Pseudogenes are Abundant in Human Serum and Plasma

Small noncoding RNAs carry out a variety of functions in eukaryotic cells, and in multiple species they can travel between cells, thus serving as signaling molecules. In mammals multiple small RNAs have been found to circulate in the blood, although in most cases the targets of these RNAs, and even their functions, are not well-understood. YRNAs are small (84-112 nt) RNAs with poorly characterized functions, best known because they make up part of the Ro ribonucleoprotein autoantigens in connective tissue diseases. In surveying small RNAs present in the serum of healthy adult humans, we have found YRNA fragments of lengths 27 nt and 30-33 nt, derived from the 5′ ends of specific YRNAs and generated by cleavage within a predicted internal loop. Many of the YRNAs from which these fragments are derived, were previously annotated only as pseudogenes, or predicted informatically. These 5′ YRNA fragments make up a large proportion of all small RNAs (including miRNAs) present in human serum. They are also present in plasma, are not present in exosomes or microvesicles, and circulate as part of a complex with a mass between 100 and 300 kDa. Mouse serum contains far fewer 5′ YRNA fragments, possibly reflecting the much greater copy number of YRNA genes and pseudogenes in humans. The processing and secretion of specific YRNAs to produce 5′ end fragments that circulate in stable complexes are consistent with a signaling function.

Small noncoding regulatory RNAs, including miRNAs, siRNAs, piRNAs, and others, have been the focus of much recent interest, not only because they are crucial for a wide range of biological functions, but also because they are involved in the pathology of cancer and many other human diseases (Esteller M., Nature reviews Genetics 12: 861-874, 2011; Joshua-Tor L, and Hannon G J., Cold Spring Harbor perspectives in biology 3: a003772, 2011; Martens-Uzunova et al., Cancer letters 2013; Okamura K., Wiley interdisciplinary reviews RNA 3: 351-368, 2012; Wery et al., Wiley interdisciplinary reviews Systems biology and medicine 3: 728-738, 2011; Zhang C., Current opinion in molecular therapeutics 11: 641-651, 2009; Zhang et al., Plant physiology, 150: 378-387, 2009). Although miRNAs in particular have been found to have broad biological roles, next generation sequencing has revealed new small RNA types with uncertain functions. Well-described small noncoding RNAs such as tRNAs, snoRNAs, and YRNAs have been found to give rise to smaller RNA species (Dhahbi et al., BMC genomics 14: 298, 2013; Kapranov et al., Science, 316: 1484-1488, 2007; Rother and Meister, Biochimie, 93: 1905-1915, 2011; Tuck and Tollervey, Trends in Genetics, 27: 422-432, 2011); although in many cases the functions of the noncoding RNAs that undergo processing into smaller RNAs are known, the functions of their smaller derivatives remain poorly understood. Intracellular 5′ tRNA halves have been shown to be cleaved by the ribonuclease angiogenin in response to stress and infections; the generated 5′ tRNA halves promote assembly of stress granules carrying stalled preinitiation complexes, and inhibit mRNA translation (Gong et al., BMC infectious diseases, 13: 285, 2013; Ivanov et al., Molecular Cell, 43: 613-623, 2011; Saikia et al., The Journal of biological chemistry, 2012). Some snoRNA-derived RNAs exhibited miRNA-like regulatory activity, while the expression levels of other snoRNA-derived RNAs are altered in cancer (Martens-Uzunova et al., Oncogene 31: 978-991, 2012). It has been proposed that snoRNA-derived RNAs may act as tumor suppressors and oncogenes (Martens-Uzunova et al, Cancer letters, 2013). Human YRNA-derived fragments were first detected in cells exposed to apoptotic stimuli (Rutjes et al., The Journal of biological chemistry, 274: 24799-24807, 1999). They were later observed in solid tumors (Meiri et al., Nucleic acids research, 38: 6234-6246, 2010; Schotte et al., Leukemia, 23: 313-322, 2009) and in cultured cells as a response to the chemical stressor poly(I:C) (Nicolas et al., FEBS letters, 586: 1226-1230, 2012).

In both plants and animals, some small RNAs are able to travel between tissues within an organism, thus transferring their functions to other cells. There has been much recent interest in specific miRNAs circulating in the plasma and serum, and some evidence that these can be taken up by cells and alter gene expression; there is also interest in the possibility that they can be markers of specific disease states, particularly cancer (Allegra et al., International journal of oncology, 41: 1897-1912, 2012; Etheridge et al., Mutation Research 717: 85-90, 2011; Zen K, and Zhang C Y, Medicinal research reviews, 32: 326-348, 2012). Using deep sequencing, we recently demonstrated that the levels of many miRNAs circulating in the mouse are increased with age, and that these increases can be antagonized by calorie restriction (Dhahbi et al, Aging, 5: 130-141, 2013). The genes targeted by this set of age-modulated miRNAs are predicted to regulate biological processes directly relevant to the manifestations of aging, and the miRNAs themselves have been linked to diseases associated with old age.

We recently reported a novel class of circulating small RNAs, 5′ tRNA halves, which prior to our report were described only as stress-induced inhibitors of translation initiation in cultured cells (Dhahbi et al., BMC genomics, 14: 298, 2013). We found that the 5′ tRNA halves are concentrated in hematopoietic and lymphoid tissues, and present in other tissues at very low levels, suggesting that they may be processed in blood cells and released into the blood. Our findings imply that 5′ tRNA halves function on an organismal rather than merely a cellular level. Moreover, they likely function in a context much broader than cellular stress or infection: we find circulating 5′ tRNA halves in unstressed conditions. Changes in their expression with age are also consistent with a broader physiologic role, and it is particularly interesting that these changes are partially mitigated by calorie restriction.

The subject of this report is yet another derivative of a known class of small noncoding RNAs, the YRNAs. They are a largely unexplored noncoding RNA species that are transcribed by RNA polymerase III from four YRNA genes in man (hY1, hY3, hY4 and hY5), and two genes in mice (mY1 and mY3) (Wolin and Steitz, Cell, 32: 735-744, 1983). The sizes of the human YRNAs are 112 nt (hY1), 101 nt (hY3), 98 nt (hY4), and 84 nt (hY5). In addition to the annotated genes, the human genome carries a very large number of YRNA sequences that have been annotated as pseudogenes, while the mouse has few or none (Perreault et al., Nucleic acids research, 33: 2032-2041, 2005; Perreault et al., Molecular biology and evolution, 24: 1678-1689, 2007). YRNAs are components of Ro ribonucleoproteins (Ro RNPs), which are clinically significant autoantigens that are recognized by antibodies in patients with connective tissue diseases (Bouffard et al., The Journal of rheumatology, 23: 1838-1841, 1996; Lerner et al., Science, 211: 400-402, 1981; Reed et al., J Immunol, 191: 110-116, 2013). Although YRNAs are reported to function in chromosomal DNA replication and quality control of noncoding RNA (Sim and Wolin, Wiley interdisciplinary reviews RNA, 2: 686-699, 2011), the function of YRNA-derived fragments has yet to be elucidated. Here we report the presence of abundant cell-free YRNA-derived fragments circulating as large complexes in human serum and plasma. These fragments are derived mostly from the 5′ ends of YRNAs and seem to rise from cleavage of YRNAs at a predicted internal loop to produce what we term “5′ YRNA fragments.”

### Materials and Methods

### Collection of Blood and Separation of Serum and Plasma.

Blood samples were collected from 5 adult women between 30 and 57 years of age, after obtaining informed consent. To obtain serum samples, blood was collected in BD Vacutainer SST tubes (#367985, BD, Franklin Lakes, N.J.), incubated for 30 min at room temperature to allow coagulation, and centrifuged at 5,000 g for 10 min. To obtain plasma samples, blood was collected in BD K2 EDTA Spray-Dried tubes (#366643, BD, Franklin Lakes, N.J.) or in tubes containing sodium heparin (5.5 mg/ml), transferred to Leucosep tubes (#227290P, Grenier Bio One, Monroe, N.C.) and centrifuged at 800 g for 15 min at room temperature. Serum and plasma supernatants were transferred to new tubes, centrifuged at 16,000 g for 15 min to remove any residual cells and cell debris, and stored at −80° C. before use. Blood samples were also collected from 5 male B6C3F1 mice (Charles River Laboratories) at 7 months of age. Immediately after collection, blood was transferred to BD Microtainer tubes (#365967, BD, Franklin Lakes, N.J.), and processed as described above for the human blood samples to prepare mouse serum.

### RNA Isolation and Small RNA Library Construction.

Isolation of total RNA, including small RNA, was performed with the miRNeasy kit (#217004, Qiagen, Hilden, Germany) according to the manufacturer's protocol except for the following alterations: 1 mL of Qiazol reagent was mixed with 0.2 mL serum or plasma, the entire aqueous phase was loaded onto a single column from the MinElute Cleanup Kit (#74204, Qiagen, Hilden, Germany), and RNA was eluted in 20 μL of RNase-free water. One fourth (5 μL) of the RNA isolated from each serum or plasma samples was used to construct sequencing libraries with the Illumina TruSeq Small RNA Sample Prep Kit (#RS-200-0012, Illumina, San Diego, Calif.), following the manufacturer's protocol. Briefly, 3′ and 5′ adapters were sequentially ligated to small RNA molecules and the obtained ligation products were subjected to a reverse transcription reaction to create single stranded cDNA. To selectively enrich those fragments that have adapter molecules on both ends, the cDNA was amplified with 15 PCR cycles using a common primer and a primer containing an index tag to allow sample multiplexing. The amplified cDNA constructs were gel purified, and validated by checking the size, purity, and concentration of the amplicons on the Agilent Bioanalyzer High Sensitivity DNA chip (#5067-4626, Genomics Agilent, Santa Clara, Calif.). The libraries were pooled in equimolar amounts, and sequenced on an Illumina HiSeq 2000 instrument to generate 50 base reads.

### Mapping and Annotation of Sequencing Reads.

Sequencing reads were pre-processed with FASTX-Toolkit (hannonlab.cshl.edu) to trim the adaptor sequences, and discard low quality reads. The filtered reads were mapped to the human (hg19) or mouse (mm10) genomes with Bowtie version 0.12.8 (Langmead et al., Genome biology 10: R25, 2009) using the “end-to-end k-difference (−v)” alignment mode and allowing 2 or 0 mismatches. In addition, this mode of alignment was combined with options that define which and how many alignments should be reported: the option “−k 1-best” instructed Bowtie to report only the best alignment if more than one valid alignment exists, while the option “−m 1” instructed Bowtie to refrain from reporting any alignments for reads having multiple reportable alignments Annotation of the mapped sequencing reads was performed with BEDTools (Quinlan et al., Bioinformatics 26: 841-842, 2010) using noncoding RNAs from Ensembl GRCh37 release 70, miRNAs from miRBase and tRNAs from Genomic tRNA Database (Chan and Lowe, Nucleic acids research 37: D93-97, 2009).

### Northern Blot Analysis.

RNAs analyzed with Northern blots were extracted from normal or UV-irradiated U2OS cells (# HTB-96, ATCC, Manassas, Va.) and from fractionated human serum. Before RNA extraction, some serum samples were centrifuged at 110,000 g for 2 hours, followed by separation of supernatant and pellet fractions, and others were separated into concentrate and filtrate fractions by ultrafiltration through Vivaspin 2 columns (GE Healthcare) with 100 or 300 kDa MW cut-off. Total RNA including small RNA was isolated from cell pellets or serum fractions with the miRNeasy kit (Qiagen). RNAs were separated on 15% denaturing polyacrylamide gels, transferred, and fixed to a membrane by chemical cross-linking (Pall GS, and Hamilton A J., Nature protocols 3: 1077-1084, 2008). Blots were hybridized overnight at 42° C. in ULTRAhyb-Oligo Buffer (Invitrogen) with the following ³²P-5′-end labeled oligonucleotide probes against the 5′ end (5′-AGTTCTGATAACCCACTACCATCGGACCAGCC; SEQ ID NO:8), or 3′ end (5′-AGCCAGTCAAATTTAGCAGTGGGGGGTTGTAT; SEQ ID NO:9) of RNY4. Membranes were washed twice with 2×SSC at 42° C., 0.1% SDS for 30 minutes, and exposed to X-ray films for detection of signals

### Results

### Sequencing and Computational Analysis of Small RNAs Circulating in Human Serums.

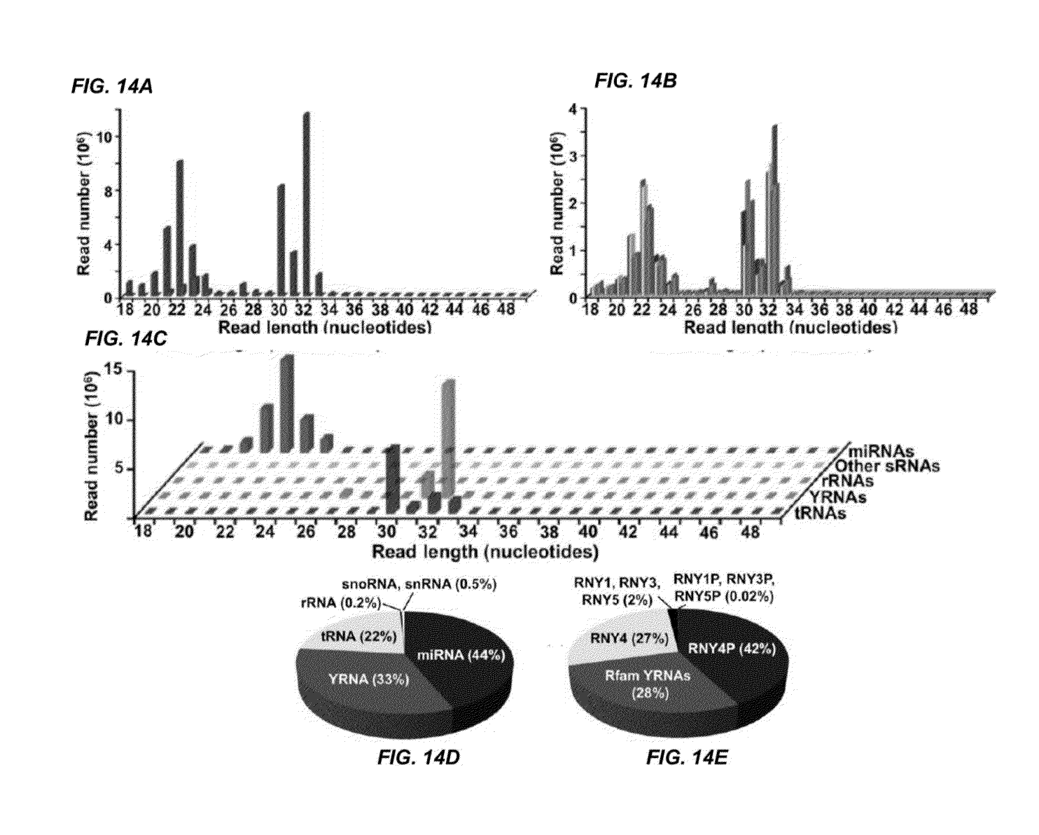

We used RNA-Seq (Illumina reads of 50 nt) to characterize small RNAs circulating in human serum, using indexed libraries to distinguish reads from each serum sample. A combined total of 58,203,901 pre-processed sequencing reads was obtained from five human serum samples. The pooled sequencing reads were mapped to the human genome with Bowtie using parameters that align reads according to the end-to-end k-difference policy, allowing two mismatches and reporting only the best alignment if more than one valid alignment exists (Langmead et al., Genome biology 10: R25, 2009). This analysis generated a dataset of 51,887,820 mapped reads (89.15%), ranging in size from 18 to 49 nt. When reads with more than one alignment were discarded, the size distribution of the mapped reads revealed an expected peak at 20-24 nt consistent with the size of miRNAs (FIG. 14A, red bars). When multiple reportable alignments are allowed, two more peaks emerge: a major peak at 30-33 nt and a minor peak at 27 nt (FIG. 14A, blue bars). The same pattern was observed when reads from the individual serum samples were mapped to the human genome allowing two mismatches and multiple reportable alignments (FIG. 14B).

Annotation of the mapped sequencing reads revealed that, as expected, reads in the 20-24 nt peak were derived from miRNAs (FIG. 14C). Reads in the 27 nt peak map to YRNA genes, while the 30-33 nt peak consists of reads mapping to YRNA genes, and to a lesser extent to tRNA genes (FIG. 14C). Further annotation analysis showed that of the total 51,887,820 reads that mapped to the human genome, 45,890,222 (88%) align to known small RNAs, of which 44% were annotated as miRNAs, 33% as YRNAs, and 22% as tRNAs (FIG. 14D). The remaining <1% of reads mapped to sequences annotated as rRNA, snRNA and snoRNA. We have previously characterized 5′ tRNA halves circulating as large nucleoprotein complexes in the mouse (Dhahbi et al., BMC genomics 14: 298, 2013), and the tRNAs sequenced here appear to be the human correspondent of those tRNA fragments. Here we focus on the reads annotated as YRNAs.

Most circulating small RNAs that align to YRNA genes are derived from the RNY4 gene and its pseudogenes (FIG. 1E). The YRNA genes used in our analysis are from Ensembl GRCh37 release 70, where the YRNAs are found in the ‘misc_RNA’ category under the ‘gene_biotype’ attribute. Ensembl annotates 3 groups as YRNAs: i) four human YRNAs: RNY1, RNY3, RNY4, and RNY5; ii) pseudogenes originating from the four human YRNAs; and iii) a group of predicted YRNAs from the Rfam database. Among the serum-derived sequence reads that align with YRNAs, 27% map to RNY4, while 42% map to the RNY4 pseudogenes (FIG. 14E). Only 2% of YRNA reads map to RNY1, RNY3, and RNY5 combined, while an additional 0.02% map to the pseudogenes of RNY1, RNY3, and RNY5 combined (FIG. 14E). A further 28% of YRNA reads map to the YRNAs predicted in Rfam (FIG. 14E). These results indicate that the 4 human YRNAs are present in the circulation in non-random proportions; genes annotated as YRNA pseudogenes, and Rfam-predicted YRNAs, are also expressed.

Characterization of Circulating Small RNAs that Align to YRNAs.

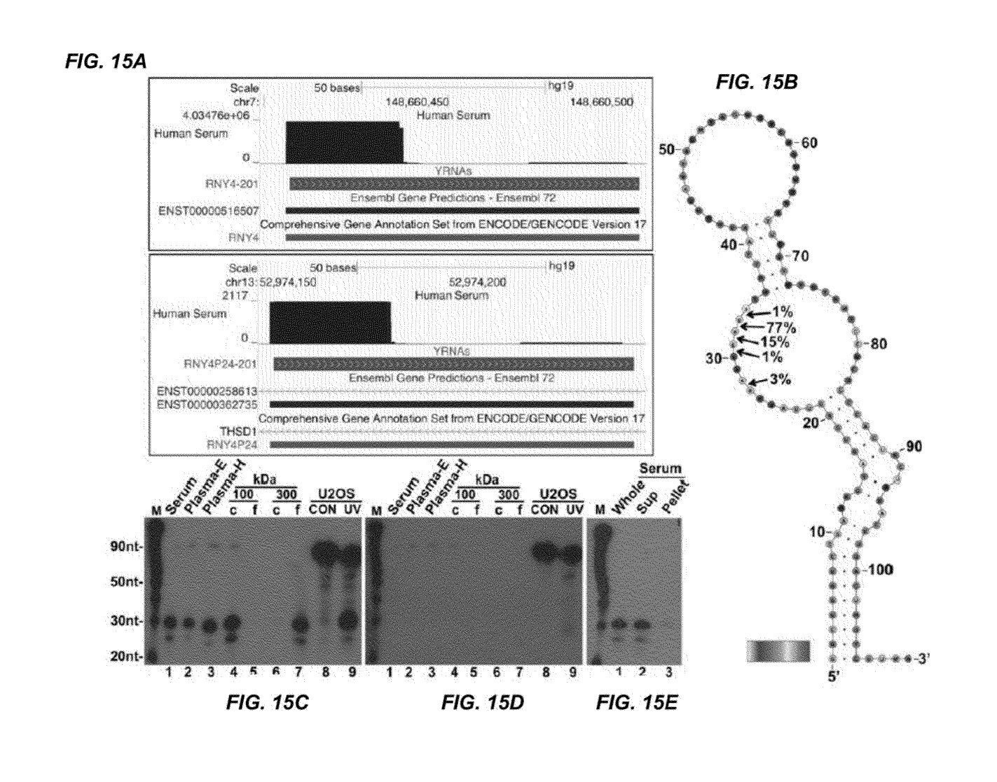

The serum-derived sequencing reads that align to YRNA genes are either 27 nt or 30-33 nt in size, while the size of full length YRNAs is 84-112 nt. We asked if the YRNA reads were the products of random fragmentation of full-length YRNAs, or alternatively show evidence of processing to produce specific fragments. We examined the alignment of YRNA reads to the genes from which they were transcribed, and annotated them based on their overlap with 5′ or 3′ ends of the genes. This analysis revealed that >95% of the YRNA-derived reads align with the 5′ end of YRNA; this is exemplified in FIG. 15A for the transcripts ENST00000516507 and ENST00000362735 encoded by the RNY4 gene and the RNY4P24 pseudogene, respectively. This alignment places the 3′ end of the fragment in an internal loop of a predicted schematic YRNA structure (FIG. 15B). We note also that the lengths of reads mapping to YRNAs vary, and that the proportions of each length are distinctly different [FIG. 15B: 27 nt (3%), 30 nt (1%), 31 nt (15%), 32 nt (77%), and 33 nt (1%)]; thus cleavage seems to occur at varying rates at different sites in the predicted internal loop. This evidence indicates that 5′ YRNA fragments circulating in the serum are generated by cleavage in the internal loop of full-length YRNA transcripts.

Northern blotting confirms the presence 5′ YRNA fragments in human serum and plasma. A probe specific for the 5′ end of RNY4 detected a major band migrating near the 30 nt RNA marker, and a minor band at ˜27 nt (FIG. 15C, lane 1). This verifies the sequencing data and confirms the presence of 5′ YRNA fragments circulating in the bloodstream in a stable cell-free form. The two-band pattern was also detected in an equal volume of EDTA or heparin plasma obtained from the same blood sample as the serum (FIG. 15C, lanes 2-3). This result indicates that the chelating agent EDTA used as an anticoagulant in preparing the plasma sample does not affect the circulating complexes containing the 5′ YRNA fragments. This is in contrast to circulating complexes containing the 5′ tRNA halves, which we found to be destabilized by EDTA (Dhahbi et al., BMC genomics 14: 298, 2013).

As a positive control for detection of YRNA fragments by Northern blotting, we included RNA from U2OS cells exposed to UV irradiation, which is known to strongly induce apoptosis. Cleavage of YRNAs, with generation of stable cellular YRNA-derived fragments, has been observed after exposure of cells to apoptotic stimuli (30). RNA extracted from U2OS cells treated with UV produced the same two bands present in serum (FIG. 15C, lanes 8-9), further validating the 5′ YRNA fragments identified by deep sequencing of circulating small RNAs. No significant bands migrating with the 30 nt RNA marker were detected when the same Northern blot was probed for the 3′ end of RNY4 (FIG. 15D). These results indicate that YRNA fragments found circulating in the blood are highly similar to fragments produced during apoptosis.

We next asked if the 5′ YRNA fragments are free, or contained within circulating exosomes or microvesicles. We Northern blotted RNA extracted from pellet and from supernatant after ultracentrifugation of human serum at 110,000 g for 2 hours. A probe for the 5′ end of RNY4 detected bands (at ˜30 nt and ˜27 nt) present in the supernatant and visible only as a trace in the pellet (FIG. 15E), indicating that YRNA is not circulating in an exosome or microvesicle. Because the YRNA fragments are stable in the circulation, but not encapsulated in exosomes, they are most likely complexed to carrying factors (e.g., proteins that protect them from degradation). To determine the size range of the putative complexes carrying the YRNA fragments in the serum, we Northern blotted RNA extracted from concentrate or filtrate fractions after ultrafiltration of human serum samples through Vivaspin 2 columns with 100 or 300 kDa MW cut-off. A probe for the 5′ end of RNY4 detected the familiar two bands (at ˜30 nt and ˜27 nt) in the concentrate of 100 kDa MW cut-off, and in the filtrate of 300 kDa MW cut-off (FIG. 15C, lanes 4-7), while a probe for the 3′ end of RNY4 did not detect any significant signal (FIG. 15D, lanes 4-7). This result suggests that 5′ YRNA fragments circulate as complexes with a mass between 100 and 300 kDa.

Human Serum and EDTA Plasma have Similar Profiles of Circulating 5′ YRNA Fragments.

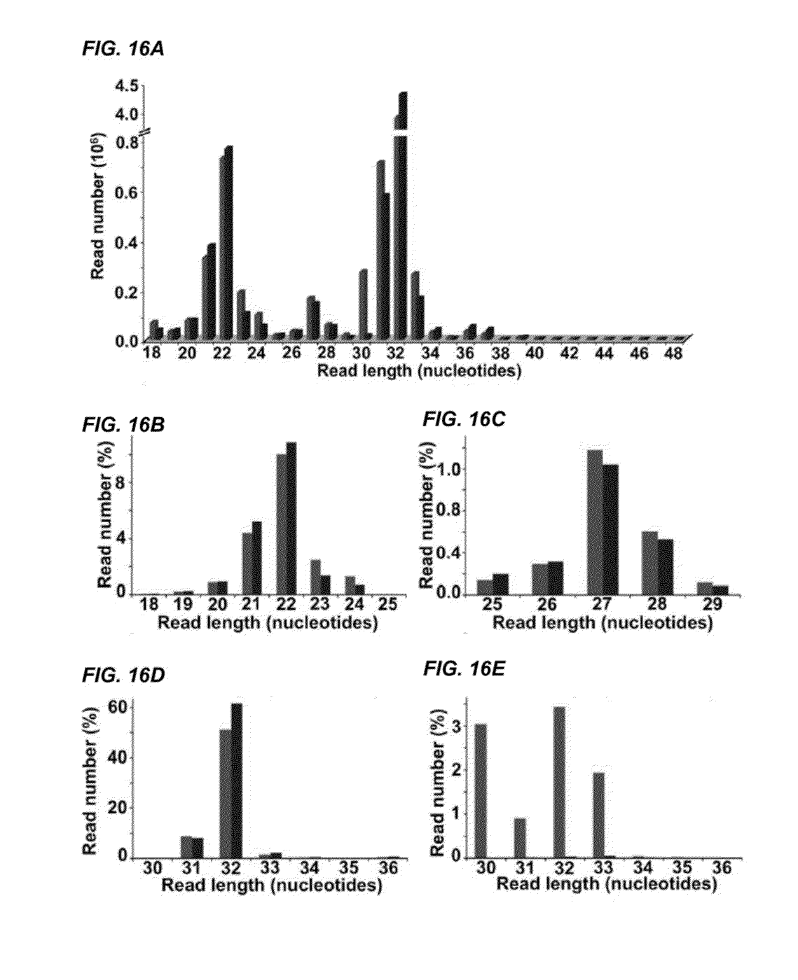

We asked if the same 5′ YRNA fragments are present in both serum and plasma. We prepared serum and plasma from blood collected from the same individual at the same time; plasma was prepared from blood treated with the anticoagulant EDTA, and serum from coagulated blood. Sequencing of small RNAs extracted from equal amounts of serum and EDTA plasma shows that plasma displays the same peak pattern (20-24 nt, 27 nt and 30-33 nt peaks) found in serum, with the exception that reads of 30 nt are significantly under-represented in the EDTA plasma when compared to serum (FIG. 16A).

Comparison of the annotations of the sequencing reads revealed that miRNAs map to the 20-24 nt peak approximately equally in serum and EDTA plasma (FIG. 16B). YRNAs also map to the 27 nt and 30-33 nt peaks in both serum and EDTA-plasma (FIG. 16C-D). We previously observed that 5′ tRNA halves circulate in the mouse as complexes that are disrupted by EDTA treatment, so that they are not present in EDTA plasma (7). Consistent with this, tRNAs that map to the 30-33 nt peak in the serum sample (FIG. 14C), were barely detected in the EDTA plasma when compared to the serum (FIG. 16E). This accounts for the significant under-representation of 30 nt reads in the EDTA plasma noted above (FIG. 16A). This result indicates that, in contrast to circulating complexes carrying 5′ tRNA halves, the circulating complexes of 5′ YRNA fragments are not sensitive to chelation of ions.

5′ YRNA Fragments are Much More Abundant in Human than in Mouse Serum.

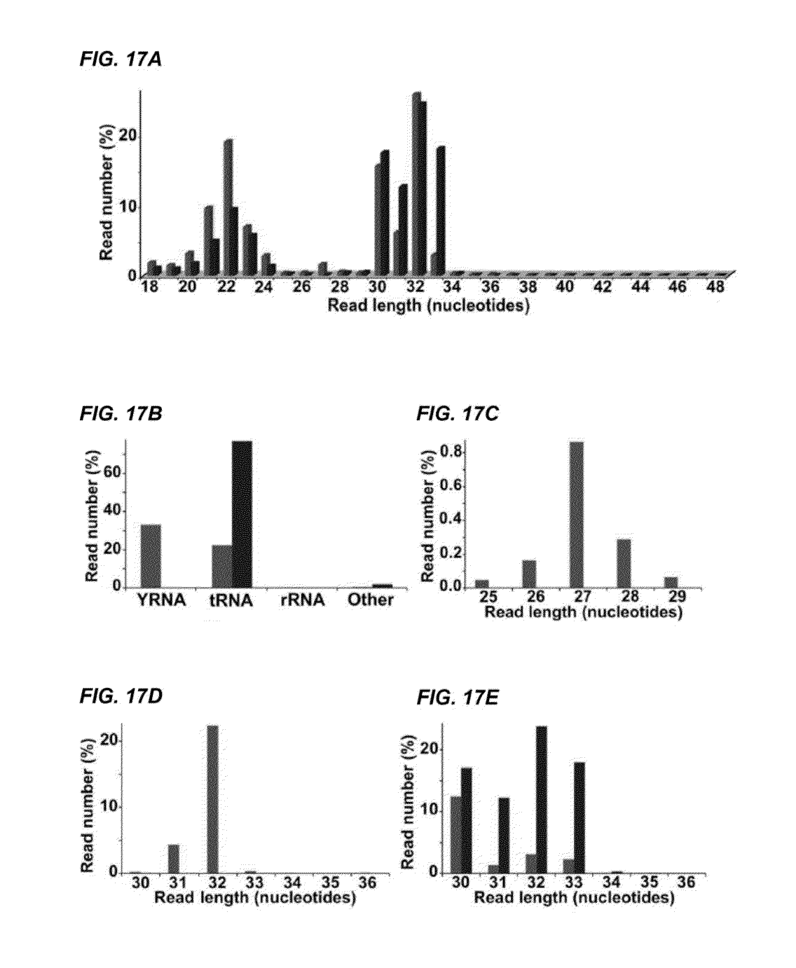

We sequenced five mouse serum samples to obtain a combined total of 71,725,136 pre-processed sequencing reads. Alignment to the mm10 mouse genome with Bowtie using the end-to-end k-difference policy while allowing two mismatches and reporting only the best alignment if more than one valid alignment exists (16), generated a dataset of 62,111,449 mapped reads (86.6%), ranging from 18 to 49 nt. Comparison of the length distribution revealed that both human and mouse serum display 20-24 nt and 30-33 nt peaks (FIG. 17A). However, reads of length 27 nt are significantly less abundant in mouse than in human serum (FIG. 17A). Comparison of the annotation of the sequencing reads from the human and mouse sera revealed a major difference in the composition of circulating small RNAs between human and mouse (FIG. 17B-E). The 5′ YRNA fragments are abundant in human serum, but scarce in the mouse, whereas the 5′ tRNA halves are significantly more abundant in the mouse serum (FIG. 17B). While abundant 26-28 nt and 30-33 nt YRNA reads are present in human serum, they are almost absent from mouse serum (FIG. 17C-D). Instead, tRNAs make up the bulk of 30-33 nt reads in mouse serum (FIG. 17E).

### Discussion

While surveying the profiles of cell-free small RNAs circulating in human blood, we identified abundant small RNAs derived from YRNAs, a class of small noncoding RNAs which complex with Ro protein in the cytoplasm, but as yet have incompletely characterized functions. We obtained 45,890,222 sequencing reads aligning to known small RNAs and found that 33% of these reads were annotated as YRNAs (FIG. 14D). Furthermore, >95% of the sequencing reads that map to YRNA genes are 27 nt or 30-33 nt long and align with the 5′ end of YRNAs, indicating that they were produced by cleavage of the parent YRNA. Northern blotting (FIG. 15C-E) confirms the presence of 5′ YRNA fragments circulating in the bloodstream in a stable cell-free form.

The serum YRNAs are derived from a subset of YRNA genes, many of them previously annotated as pseudogenes. While 27% of all sequencing reads that align with YRNAs were derived from RNY4, only 2% mapped to RNY1, RNY3, and RNY5 combined (FIG. 14E). This finding indicates that the 4 human YRNAs are disproportionately represented in the circulation, implying a type-specific biogenesis and/or release of the circulating 5′ YRNA fragments. The Rfam database includes a group of predicted YRNAs assembled from noncoding RNAs with conserved RNA secondary structure; 28% of our YRNA reads map to Rfam-predicted YRNAs (FIG. 14E), supporting the validity of the Rfam predictions.

More interestingly, 42% of the sequencing reads that align with YRNAs map to pseudogenes arising from RNY4, while only 0.02% map to the pseudogenes of RNY1, RNY3, and RNY5 combined (FIG. 14E). There are 1000 YRNA pseudogenes in the human genome, while YRNA pseudogenes are very rare in the mouse genome (Perreault et al., Nucleic acids research 33: 2032-2041, 2005, Perreault et al., Molecular biology and evolution 24: 1678-1689, 2007). This result clearly indicates that RNY4 sequences in the human genome that have been annotated as pseudogenes are transcribed, and that the transcripts are processed and secreted, calling into question their annotation as pseudogenes. Because so little is known about the biological role of YRNAs in general, and nothing is known about potential function of the circulating 5′ YRNA fragments we have found, the significance of this finding is at present unclear. However, there is evidence that a class of pseudogenes that arose from human YRNAs through the L1 retrotransposition machinery may be involved in post-transcriptional regulation of genes (Perreault et al., Nucleic acids research 33: 2032-2041, 2005, Perreault et al., Molecular biology and evolution 24: 1678-1689, 2007).

The YRNA reads represent fragments processed from full length (84-112 nt) YRNAs: the sequencing runs used to generate these reads were 50 cycles, yet only reads of length 27 nt or 30-33 nt are recovered and longer species were not present (FIG. 14A-C). Despite the primary sequence divergence among YRNAs (genes and pseudogenes), their secondary structure as predicted by Varna (Darty et al., Bioinformatics 25: 1974-1975, 2009) is characterized by a large internal loop and a stem structure formed by base-pairing between the highly conserved 5′ and 3′ ends (FIG. 15B). Internal loops in YRNAs have been shown to be accessible to nucleases that cleave single-stranded RNA (Chen X, and Wolin S L., J Mol Med (Berl) 82: 232-239, 2004; Teunissen et al., Nucleic acids research 28: 610-619, 2000; van Gelder et al., Nucleic acids research 22: 2498-2506, 1994). Given the existence of a predicted internal loop, and the narrow size range of 27-33 nt of sequencing reads that map to YRNAs, we suggest that full length YRNA transcripts are cleaved in the internal loop to generate the 5′ YRNA fragments found in serum. In addition, the variety of 5′ YRNA fragment sizes indicates that full length YRNAs are cleaved at more than one site, and at varying rates, to generate the 5′ YRNA fragments found in serum (FIG. 15B).

Because clotting has the potential to release cellular components that are not present in circulating blood, we asked if the same peak pattern of small RNAs in the human serum is also present in human plasma. Sequencing analysis of small RNAs extracted from serum and EDTA plasma samples prepared from the same person revealed that YRNAs are present in equivalent amounts and types in serum and EDTA plasma (FIG. 16), demonstrating that serum 5′ YRNA fragments are not an artifact of blood clotting. We find evidence that the 5′ YRNA fragments circulate as part of a complex with a mass between 100 and 300 kDa (FIG. 15C-D). This complex is not destabilized by the chelating agent EDTA, in contrast to our previous finding that complexes carrying circulating 5′ tRNA halves are highly sensitive to EDTA.

This study points out a puzzling feature of circulating small RNAs: 5′ YRNA fragments are abundant in human serum, but scarce in the mouse (FIG. 17C-D), while the converse seems to be the case with 5′ tRNA halves (FIG. 17E). The apparent low abundance of circulating 5′ tRNA halves in human serum is to some extent a function of the high abundance of 5′ YRNA fragments: 5′ tRNA halves are present, but form a lower proportion of all small RNAs than in the mouse, where there are a few 5′ YRNA fragments. The near absence of YRNA-derived fragments in mouse serum may reflect the scarcity of YRNA gene copies, and in particular YRNA pseudogene copies, in the mouse genome, and suggests that any presumed function of the circulating 5′ YRNA fragments is not deeply conserved. While YRNA genes themselves are conserved, humans, but not mice carry a large number of YRNA pseudogenes.

Secreted miRNAs, the most extensively studied circulating small RNAs, circulate in the blood as part of microvesicles, exosomes, or apoptotic bodies, and also in association with the lipoproteins HDL and LDL, Argonaute proteins, nucleophosmin-1, and ribosomal proteins L10a and L5 (Arroyo et al., Proceedings of the National Academy of Sciences of the United States of America, 5003-5008, 2011; Turchinovich A, and Burwinkel B., RNA biology 9: 2012; Turchinovich et al., Nucleic acids research 39: 7223-7233, 2011; Vickers et al., Nature Cell Biology 13: 423-433, 2011; Wang et al., Nucleic acids research 38: 7248-7259, 2010; Zernecke et al., Science Signaling 2: ra81, 2009). Nothing is currently known about the packaging of circulating small RNAs other than miRNAs, nor is it known how small RNAs, including miRNAs, make their way out of the cell into the extracellular space. Our Northern blot analysis of RNA extracted from pellet or supernatant after ultracentrifugation of human serum indicates that circulating 5′ RNY4 fragments are not included in exosomes or microvesicles (FIG. 2E). In line with this observation, exosome encapsulation is not required for the stability of circulating miRNAs and 5′ tRNA halves. Because the YRNA fragments are stable in the circulation but not encapsulated in exosomes, they are most likely complexed to proteins that protect them from degradation. While the 5′ YRNA fragments have a predicted mass of only ˜10 kDa, our analysis indicates that they circulate as part of 100-300 kDa complexes (FIG. 2E), the nature and identity of which remain to be determined.

Currently the tissues/cells of origin of circulating small RNAs, the mechanisms by which they are delivered, and their functions in recipient cells, remain largely unknown. However, information about the properties of one type of circulating small RNAs, i.e., miRNAs, has been emerging. Vickers et al. demonstrated that circulating miRNA/HDL complexes from atherosclerotic subjects, when delivered into cultured hepatocytes, altered expression of genes with functions related to lipid metabolism, inflammation, and atherosclerosis (Vickers et al., Nature Cell Biology 13: 423-433, 2011). Extracellular miRNAs secreted by endothelial cells are reported to alter gene expression in recipient cells. miR-126 triggered the production of the chemokine CXCL12 in recipient vascular cells (Zernecke et al., Science Signaling 2: ra81, 2009) while miR-143/145 altered gene expression in co-cultured smooth muscle cells to reduce the formation of atherosclerotic lesions in the aorta of ApoE(−/−) mice (Hergenreider et al., Nature cell biology 14: 249-256, 2012). Similarly, miR-150 secreted by human blood cells and cultured monocytic THP-1 cells, reduced c-Myb expression and enhanced cell migration after delivery into HMEC-1 cells (Zhang et al., Molecular cell 39: 133-144, 2010). Thus, there is evidence that extracellular miRNAs can enter target cells and alter gene expression with significant functional consequences. This suggests that other circulating small RNAs, such as 5′ YRNA fragments and 5′ tRNA halves, may also be capable of crossing the membranes of target cells and modulating cellular functions.

Reports of YRNA-derived fragments in cells or tissues are scant. Human YRNA-derived fragments were first observed in cells exposed to apoptotic stimuli (Rutjes et al., The Journal of biological chemistry 274: 24799-24807, 1999). The apoptosis-induced YRNA fragments have small (22-25 nt) and large (27-36 nt) sizes, and remain bound to Ro after they are cleaved. However, whether these fragments are derived from the 5′ or 3′ ends, or both, was not determined; Rutjes and colleagues (Rutjes, supra) used a non-specified mixture of probes for the four human YRNAs during Northern blot analysis. The same study also showed that the cleavage of YRNA is caspase-dependent. This suggests that the nucleases that cleave YRNAs might be caspase-activated nucleases also involved in inter-nucleosomal cleavage of chromatin that results in the DNA ladder during apoptosis. Whether the 5′ YRNA fragments abundantly circulating in the bloodstream of healthy human subjects can be linked to such an apoptotic cleavage remains to be investigated.

Production of 3′ end fragments of human RNY5 was observed upon treatment of cancerous and non-cancerous cell lines with the stressor poly(I:C), a double-stranded RNA mimic immunostimulant chemical (Nicolas et al., FEBS letters 586: 1226-1230, 2012). The same study reported the presence of 3′ end fragments of human RNY5 RNA in non-stressed MCF 7 mammary adenocarcinoma cells (Nicolas, supra). Only a human RNY5 3′ end probe was used in the Northern blotting analysis in this study, and so it is not known if 5′ end fragments of human RNY5 RNA were also present in these cells. Likewise, two 25-nt fragments derived from RNY1 and RNY3 RNAs were detected in solid tumors and in normal serum (Meiri et al., Nucleic acids research 38: 6234-6246, 2010; Schotte et al., Leukemia 23: 313-322, 2009). These two small RNAs were initially classified as miRNAs, but subsequently removed from miRBase because they lacked gene regulatory activity. Larger (27-36 nt) fragments derived from YRNAs, similar to the ones reported here, were not reported in solid tumors and in normal serum, most likely because in these studies sequences whose length exceeded 17-25 nt were systematically discarded (Meiri et al., Nucleic acids research 38: 6234-6246, 2010). In another study, 28 nt YRNA fragments were found in vesicles released by immune cells, along with full length YRNAs, and full length and derivatives of SRP-RNA and vault-RNA (Nolte-'t Hoen et al., Nucleic acids research 40: 9272-9285, 2012). The vesicular small RNAs were enriched relative to cellular RNA, suggesting their selective release into the extracellular space and potential regulatory functions in target cells.

In this study, we have identified an abundant (comparable to miRNA) class of small RNA circulating in human blood, derived largely from genomic sequences annotated as YRNA pseudogenes. Taken together, the evidence discussed here indicates a potential for a variety of functions for 5′ YRNA fragments.

### Example 3

### Extracellular tRNA- and YRNA-Derived Fragments as Disease Biomarkers

The development of non-invasive specific biomarkers for early detection of cancer is key for effective therapeutic and preventive approaches to confront the worldwide morbidity and mortality of cancer and its rising financial burden. Circulating miRNAs are emerging as novel blood-based markers for the detection of human cancers, especially at an early stage. More recently, other small non-coding small RNAs were detected in plasma and serum, offering potential as a new class of biomarkers for diseases. Non-coding RNAs, with well known functions, undergo processing into smaller RNAs, in particular, tRNA is processed into tRNA fragments which were shown to function as inhibitors of translation initiation in response to stress in cultured cells. We recently reported the presence of tRNA- and YRNA-derived fragments in serum/plasma where they circulate as a component of a stable macromolecular complex. We found that the abundance of 5′ tRNA halves in the serum changes with age and calorie restriction, strongly suggesting that they are a novel form of signaling molecule, and thus, could serve as markers of health and disease states. YRNA-derived fragments were detected in MCF7 mammary adenocarcinoma cells and found significantly induced upon treatment of cancerous and non-cancerous cell lines with the stressor poly(I:C), a double-stranded RNA mimic immunostimulant chemical.

Here, we used high-throughput sequencing of small RNAs to perform genome-wide measurements of the serum levels of tRNA and YRNA fragments from 5 healthy female controls and 5 female patients with breast cancer. The analysis revealed that breast cancer is associated with significant differences in the abundance of circulating noncoding small RNAs derived from tRNAs and YRNAs (Tables 4 and 5). The observed differences in the levels of the circulating YRNA- and tRNA-derived fragments are linked to the presence of cancer. Thus, the profile of these fragments in serum, plasma, and other body fluids can be used new minimally invasive cancer markers.

TABLE 4
Breast cancer-associated changes in the serum levels of 5′ tRNA halves.

| | Normal | | | |
|---|---|---|---|---|
| tRNA¹ | (cpm)² | Tumor (cpm)² | FC³ | P-value |
| Arg-CCG | | | | |
| chr17: 66016013-66016085 | 49 | 145 | 3 | 0.003 |
| Arg-CCT | | | | |
| chr7: 139025446-139025518 | 23 | 47 | 2 | 0.006 |
| Arg-TCT | | | | |
| chr1: 159111401-159111474 | 6 | 17 | 3 | 0.005 |
| chr1: 94313129-94313213 | 1714 | 4033 | 2 | 0.004 |
| chr9: 131102355-131102445 | 6 | 13 | 2 | 0.007 |
| chr17: 8024243-8024330 | 12 | 22 | 2 | 0.018 |
| Asn-GTT | | | | |
| chr1: 148248115-148248188 | 26 | 72 | 3 | 0.008 |
| Cys-GCA | | | | |
| chr4: 124430005-124430076 | 85 | 164 | 2 | 0.008 |
| chr17: 37023898-37023969 | 553 | 1073 | 2 | 0.010 |
| chr17: 37025545-37025616 | 87 | 181 | 2 | 0.003 |
| chr17: 37309987-37310058 | 83 | 163 | 2 | 0.007 |
| chr17: 37310744-37310815 | 80 | 163 | 2 | 0.006 |
| Gln-TTG | | | | |
| chr6: 145503859-145503930 | 3 | 7 | 2 | 0.036 |
| Gly-TCC | | | | |
| chr1: 161432166-161432237 | 2 | 5 | 2 | 0.016 |
| Leu-AAG | | | | |
| chr5: 180528840-180528921 | 5 | 12 | 2 | 0.007 |
| chr14: 21078291-21078372 | 2 | 5 | 2 | 0.023 |
| Pro-TGG | | | | |
| chr16: 3234133-3234204 | 134 | 200 | 1 | 0.031 |
| Ser-GCT | | | | |
| chr6: 26305718-26305801 | 33 | 55 | 2 | 0.032 |
| chr6: 27265775-27265856 | 3 | 7 | 2 | 0.019 |
| Trp-CCA | | | | |
| chr6: 26331672-26331743 | 23 | 39 | 2 | 0.041 |
| Val-AAC | | | | |
| chr3: 169490018-169490090 | 15896 | 30605 | 2 | 0.017 |
| chr5: 180591154-180591226 | 16231 | 31153 | 2 | 0.017 |
| chr5: 180596610-180596682 | 16125 | 31093 | 2 | 0.016 |
| chr5: 180615416-180615488 | 3021 | 5485 | 2 | 0.021 |
| chr5: 180645270-180645342 | 4304 | 8291 | 2 | 0.016 |
| chr6: 27618707-27618779 | 4259 | 8194 | 2 | 0.017 |
| chr6: 27648885-27648957 | 4344 | 8315 | 2 | 0.017 |
| chr6: 27721179-27721251 | 4232 | 8154 | 2 | 0.017 |
| Val-CAC | | | | |
| chr1: 149298555-149298627 | 4331 | 8279 | 2 | 0.017 |
| chr1: 149684088-149684161 | 4391 | 8347 | 2 | 0.018 |
| chr1: 161369490-161369562 | 4414 | 8422 | 2 | 0.015 |
| chr5: 180524070-180524142 | 16273 | 31118 | 2 | 0.017 |
| chr5: 180529253-180529325 | 4466 | 8487 | 2 | 0.018 |
| chr5: 180600650-180600722 | 16731 | 31644 | 2 | 0.018 |
| chr5: 180649395-180649467 | 4395 | 8333 | 2 | 0.017 |
| chr6: 26538282-26538354 | 16594 | 31522 | 2 | 0.018 |
| chr6: 27173867-27173939 | 272 | 516 | 2 | 0.020 |
| chr6: 27248049-27248121 | 8352 | 17964 | 2 | 0.018 |
| chr6: 27696327-27696399 | 25 | 45 | 2 | 0.044 |
| Val-TAC | | | | |
| chr6: 27258405-27258477 | 200 | 354 | 2 | 0.035 |
| Asp-GTC | | | | |
| chr6: 27471523-27471594 | 94 | 56 | −2 | 0.039 |
| chr12: 125411891-125411962 | 29 | 15 | −2 | 0.020 |
| chr12: 125424193-125424264 | 31 | 16 | −2 | 0.015 |
| chr17: 8125556-8125627 | 30 | 14 | −2 | 0.009 |
| Lys-TTT | | | | |
| chr6: 27559593-27559665 | 115 | 61 | −2 | 0.016 |
| chr6: 28918806-28918878 | 4322 | 2407 | −2 | 0.042 |

¹tRNA isoacceptor identity with corresponding genomic positions in the human hg19 genome.
²Average tRNA read count for the indicated experimental group reported as counts per million (cpm) reads in the sequenced library.
³Fold change calculated by EdgeR from comparison between the normal and breast cancer serum samples.

TABLE 5
Breast cancer-associated changes in the serum levels of YRNA-derived fragments.

| Y_RNA¹ | Genomic coordinates | Normal (cpm)² | Tumor (cpm)² | FC³ | P-value | End⁴ |
|---|---|---|---|---|---|---|
| Y_RNA.400 | chr8:98784541-98784653 | 7.4 | 4.1 | -1.8 | 0.014 | 5′ |
| Y_RNA.353 | chr1:155092966-155093074 | 7.8 | 4.6 | -1.7 | 0.041 | 5′ |
| Y_RNA.31 | chrX:135653864-135653974 | 103.4 | 63.8 | -1.6 | 0.024 | 5′ |
| Y_RNA.112 | chr3:164840501-164840611 | 108.4 | 71.2 | -1.5 | 0.039 | 5′ |
| Y_RNA.367 | chrX:19394892-19394993 | 4.5 | 8.3 | 1.8 | 0.014 | 5′ |
| Y_RNA.639 | chr14:56535161-56535245 | 9.9 | 6.5 | -1.5 | 0.035 | 5′ |
| Y_RNA.166 | chr20:16651286-16651387 | 10.4 | 18.4 | 1.8 | 0.014 | 3′ |
| Y_RNA.597 | chr2:206890317-206890421 | 20.2 | 35.6 | 1.8 | 0.029 | 3′ |
| Y_RNA.535 | chr12:42848522-42848623 | 25.0 | 43.3 | 1.7 | 0.013 | 3′ |
| Y_RNA.180 | chr15:59867827-59867922 | 10.8 | 18.2 | 1.7 | 0.044 | 3′ |
| Y_RNA.168 | chr14:100049354-100049455 | 28.3 | 47.3 | 1.7 | 0.007 | 3′ |
| Y_RNA.450 | chr6:34789222-34789319 | 14.9 | 24.5 | 1.6 | 0.017 | 3′ |
| Y_RNA.212 | chr11:107955640-107955741 | 24.5 | 40.1 | 1.6 | 0.024 | 3′ |
| Y_RNA.511 | chr11:64063509-64063610 | 21.8 | 35.3 | 1.6 | 0.022 | 3′ |
| Y_RNA.481 | chr15:30965953-30966046 | 20.4 | 33.0 | 1.6 | 0.021 | 3′ |
| Y_RNA.505 | chr15:52454948-52455049 | 21.2 | 34.0 | 1.6 | 0.040 | 3′ |
| Y_RNA.148 | chr6:106902703-106902804 | 26.8 | 43.0 | 1.6 | 0.032 | 3′ |
| Y_RNA.469 | chr20:431307-431406 | 14.1 | 22.6 | 1.6 | 0.037 | 3′ |
| Y_RNA.170 | chr4:158689165-158689265 | 10.0 | 15.6 | 1.6 | 0.031 | 3′ |
| Y_RNA.595 | chr2:113337061-113337161 | 5.7 | 8.9 | 1.6 | 0.040 | 3′ |
| RNY4P18 | chr9:113859605-113859693 | 206.0 | 328.7 | 1.6 | 0.037 | 3′ |
| RNY4P25 | chr1:151411476-151411571 | 507.1 | 777.0 | 1.5 | 0.039 | 3′ |
| Y_RNA.218 | chr17:43148810-43148911 | 16.8 | 25.6 | 1.5 | 0.035 | 3′ |
| Y_RNA.699 | chrX:41175741-41175842 | 5.7 | 8.6 | 1.5 | 0.017 | 3′ |
| Y_RNA.492 | chr12:123252646-123252747 | 15 | 23 | 1.5 | 0.017 | 3′ |

¹YRNA identity with corresponding genomic positions in the human hg19 genome.
²Average YRNA read count for the indicated experimental group reported as counts per million (cpm) reads in the sequenced library.
³Fold change calculated by EdgeR from comparison between the normal and breast cancer serum samples.
⁴Indicates whether the sequencing reads map to the 5′ or 3′ end YRNAs.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for assessing risk of colon cancer in a human patient, said method comprising the steps of:
(1) contacting genomic DNA isolated from a colon mucosa sample from the human patient with a bisulfite, wherein the bisulfite converts unmethylated cytosines in DNA present in the sample to uracils;
(2) performing a polymerase chain reaction (PCR) to amplify a genomic DNA sequence comprising SEQ ID NO:1 using a primer consisting of SEQ ID NO:4 and a primer consisting of SEQ ID NO:5;
(3) determining methylation status of cytosine-phosphate-guanine pairs (CpGs) in the genomic sequence amplified in step (2) and comparing the number of methylated CpGs with the number of methylated CpGs in the genomic sequence from a non-cancer colon mucosa sample and processed through steps (1) to (2); and
(4) determining the human patient, whose colon mucosa sample contains more methylated CpGs in the genomic sequence amplified in step (2) compared to the number of methylated CpGs with the number of methylated CpGs in the genomic sequence from a non-cancer colon mucosa tissue sample and processed through steps (1) to (2), as having an increased risk of colon cancer compared with a human subject not diagnosed with colon cancer.

2-5. (canceled)

6. The method according to claim 1, wherein the bisulfite is sodium bisulfite.

7. The method according to claim 1, wherein step (2) comprises combined bisulfate restriction analysis (COBRA).

8-11. (canceled)

12. The method according to claim 1, wherein step (2) or (3) further comprises using a primer or probe comprising a sequence selected from the group consisting of: SEQ ID NOs:8, 9, 10, and 11.

13-23. (canceled)

24. (canceled)
